(19) United States
(12) Patent Application Publication
(10) Pub. No.: US 2026/0354377 A1
(43) Pub. Date: Nov. 12, 2026

(54) INSECTICIDAL AND ARACHNIDICIDAL COMPOSITIONS AND USES THEREOF

(71) Applicants: Michael Anthony FOLAN, Donegal Town (IE); David A. FOLAN, Donegal Town (IE)

(72) Inventors: Michael Anthony FOLAN, Donegal Town (IE); David A. FOLAN, Donegal Town (IE)

(21) Appl. No.: 19/470,118

(22) PCT Filed: Mar. 26, 2024
(86) PCT No.: PCT/IB2024/052873

§ 371 Date: Sep. 29, 2025

Related U.S. Application Data
(60) Provisional application No. 63/493,153, filed on Mar. 30, 2023.

(51) Int. Cl. A01N 37/02; A01N 25/04; A01N 37/30; A01N 43/36; A01N 57/04; A01P 7/00

(52) U.S. Cl. CPC A01N 37/02; A01N 25/04; A01N 37/30; A01N 43/36; A01N 57/04; A01P 7/00

(57) ABSTRACT

Compositions comprising reactive emulsions of free fatty acids, and membrane lipids and their use in controlling insects and/or arachnids, and in particular, hematophagous organisms which can act as vectors for a great variety of pathogens. The compositions of the present invention are also useful for the modification of a subject's physiological and/or immunological response to, for example, a bite from an hematophagous organism as well as treating and/or preventing a disease caused by any pathogen transmitted by the organism to said subject.

### BACKGROUND

The scientific term ‘Arthropod’ includes Crustaceans (lobsters and crabs), Arachnids (spiders, ticks and mites), Hexapoda (Insects—fleas, midge, mosquito and horse-fly), Chilopoda and Decapoda (centipedes and millipedes). There are a significant number from different classifications that are parasitic on humans and animals causing great discomfort and acting as vectors for infectious disease. In addition, some arthropods are parasitic on plants (Caterpillar, Aphids, Locust, Mites, Weevils and Whitefly) causing serious economic damage to crop yields.

Arthropods that are vectors for infectious disease in human and non-human animals are mainly but not exclusively restricted to midge (e.g. Culicoides impunctatus), mosquito (e.g. Aedes aegypti), hose-fly (Tabanidae), ticks (e.g. Ixodes Ricinus) and fleas (e.g. Ctenocephalides canis) although other parasitic insects including body lice (e.g. Pediculus humanis) and mites (e.g. Trombiculidae autumnalis and Sarcoptes scabiei) also present a significant health concern. It should be appreciated that the examples given are indicative and that there are a very large number of different species within each category—there are over 112 different species of Mosquito and some 1,350 different species of flea.

Arthropod borne diseases are caused by a variety of pathogens, including viruses, bacteria, fungi, and parasites (including protozoa, helminths, and ectoparasites) many of which present serious health concerns in many parts of the world and for many of these there are few safe, effective and affordable interventions.

Apart from significant immune response to midge bite saliva there are few infectious diseases transmitted to humans by midge, there are however several significant diseases affecting farm animals including Akabane virus, Schmallenberg and Blue-tongue which cause considerable animal welfare issues and economic hardship to farmers. Tularemia is a bacterial disease (Francisella tularensis) transmitted to humans and non-human animals by horse-fly bite, the same insect is noted for transmission of the parasitic worm, Loa loa, and other pathogens such as equine infectious anaemia, anthrax and trypanosomes (trypanosoma/Chagas).

Diseases transmitted by mosquito bite include but are not limited to Malaria, Dengue, yellow fever, Zika virus, Chikungunya, and Human Lymphatic Filariasis.

Ticks are recognised as the most prodigious vectors for human and non-human animal disease including but not limited to Lyme disease (Borreliosis), Tick Borne Encephalitis, Alkhurma Haemorrhagic Fever, Colorado Tick Fever, Babesiosis, Crimean-Congo Haemorrhagic Fever, Human Granulocytic Anaplasmosis, Rickettsiosis and Tick-Borne Relapsing Fever among the most notable.

The most notorious infectious disease spread by fleas is the bacterial Yersinia pestis (black death/Bubonic plaque), various Rickettsia disease and Tungiasis are transmitted by a species of burrowing flea Tunga penetrans.

Among other discomforts some species of mites cause ‘Chiggers’ a problematic skin irritation caused by Trombiculidae autumnalis, other mites including Sarcoptes scabiei cause subdermal infestations (Scabies).

In humans some species of lice are noted for infestation of hair, Pediculus Humanus Captis, while others are predominantly located on the body or in the pubic area. Of these, only body lice potentially transmit Typhus fever, but head lice are a great source of irritation particularly among children in schools where mass infestation is not uncommon.

Apart from diseases there are also significant physiological and/or immunological responses to arthropod bite saliva, for example, midge in humans and non-human animals.

Hematophagous organisms that bite mammalian skin do so to feed on fresh blood and they achieve this by incising the skin with specialised cutting mouth parts and inserting a feeding tube through which blood is sucked. They act as vectors for a great variety of pathogens, including viruses, bacteria, fungi, and parasites (including protozoa, helminths, and ectoparasites).

All mammals have elaborate defence mechanisms against skin trauma and penetration by foreign objects. The most immediate result of skin penetration by foreign objects is vasoconstriction which inhibits blood flow to the wound, followed by platelet aggregation and fibrinogen activation of fibrin to seal the wound-all of which should deny, the object, for example a hematophagous organism the blood meal it seeks.

To counteract these mammalian defence mechanisms all hematophagous organisms have evolved a complex salivary composition of macromolecular antagonists which inhibit vasoconstriction and exert anti-coagulation effect to ensure blood flow. Apart from observable gross physiological effects on blood flow and coagulation, current research shows that there is a myriad of biologically active components in insect saliva. The function of some of these components is speculative or inferred from comparative DNA sequencing but others are proven to affect mammalian immune response and in turn coincidentally promote infectivity of pathogens in the insect saliva (Lefteri D. A. et. al. PNAS Research Article, Microbiology, 2022, Vol 119, No 24).

It will be appreciated that experimental evaluation of the components of insect saliva and the characteristics of their salivary glands is challenging due to the very small amounts of salivary exudate and the microscopic nature of the glands themselves. Researchers have developed useful techniques for collecting saliva from biting insects, the most commonly used involves encouraging the insect to bite through an inert membrane (parafilm) into a reservoir of oil-usually microscope immersion oil—or into a droplet of immersion oil at the end of a capillary tube (Anderson S. L. et. al. J. Am Mosq. Control Assoc. March 2010; 26 (1); 108-111).

Insect saliva collected into immersion oil appears as discrete aqueous droplets which may be separated by centrifugation, pooled, and components separated and identified by GC, HPLC, mass spectrometry and nuclear magnetic resonance techniques. The above referenced Journal paper provides good descriptions of the myriad of components that have been isolated and their purported biological activity. It should be noted however that nearly all of the identified components are proteinaceous in nature and by definition, mostly water soluble. It is important to appreciate that any amphipathic or hydrophobic components of insect saliva will partition into the immersion oil from where they cannot be recovered without extensive solvent fractionation techniques which are not reported in current journals. Because of potential phase partitioning of lipophilic components it is likely that current understanding of the insect salivary components is incomplete.

It is reasonable to suggest that in addition to identified proteinaceous/water soluble components, insect saliva may also contain lipids, phospholipids, triglycerides and liposomal vesicles which are currently unrecognised, and which may contribute significantly to mammalian physiological response to insect saliva.

In addition to mammalian mechanisms to promote haemostasis (pro-coagulation and vasoconstriction), there is also a complex array of immune systems to identify, isolate and remove foreign material at the site of penetration of a foreign object. Mammalian immune response is normally initiated by mast cells and dendritic cells located in the dermis at the bite site. In response to bite trauma mast cells de-granulate releasing a range of locally active inflammatory agents (bradykinin and histamine) and chemoattractants (chemokines and leukotrienes) that recruit blood borne innate immune cells (neutrophils and monocytes) to the bite site. Monocytes secrete hormones that activate fibroblasts and fibrinogen while activated dendritic cells that acquire foreign matter migrate from the bite site through the lymphatic system to lymph nodes where foreign antigen is presented to naïve B and T lymphocytes generating antigen specific antibodies and T lymphocytes.

Insect saliva contains a range of active components which disrupt, suppress and frustrate the normal immune reaction described above (L. Simo et. al. The Essential Role of Tick Salivary Glands and Saliva in Tick Feeding and Pathogen Transmission: Frontiers in Cellular Infection Microbiology. Review, 22 Jun. 2017). Sialokinin, an insect tachykinin like component in mosquito saliva has been shown to cause rapid reduction in blood vessel barrier effect causing vascular leakage which enables enhanced saliva borne arbovirus infection (Lefteri D. A. et. al. PNAS Research Article, Microbiology, 2022, Vol 119, No 24.)

The normal immunological response of mammals to insect bite is considerable itching and erythema at the bite site, a reaction which is usually delayed for some time after the insect has departed. It is known that insect saliva contains histamine binding proteins which serve to delay the onset of the itch and hence the awareness of the mammal that it has been bitten, further facilitating insect feeding at the bite site. Itching is a histamine induced local nerve terminal irritation which may be directly from histamine in the insect saliva, or by a severe IgE mediated hypersensitivity reaction causing release of endogenous histamine from local mast cells (Fostini et. al. Itch (2019) 4:e19).

In commercial crop production, control of insect infestation in is critical to achieve worthwhile harvest. Insects that cause serious economic damage globally include the Cotton Bollworm, Apple maggot flies, Plum Curculio, Codling Moth, Fruit Fly, Aphids and White fly.

Chemical insecticides are the mainstay of commercial crop protection because they are relatively cheap, effective and frequently long lasting. Chemical insecticides fall into 3 categories-inorganic, organic or synthetic and may be further classified according to mode of action and whether they are inhaled, ingested or absorbed through the body surface of the target insects. Inhaled insecticides are mainly used to fumigate stored crops or seeds and include hydrogen cyanide, naphthalene and nicotine. Ingested insecticides are used to treat plants subject to being eaten by caterpillars for example and these include the arsenicals (copper, lead and calcium arsenite) and fluorine compounds such as sodium fluoride and cryolite. Contact insecticides include the naturally occurring nicotine, pyrethrum, derris root and fractions of petroleum distillation.

Modern synthetic insecticides are broadly classified into three categories:
Chlorinated hydrocarbons, of which DDT was the first and most notorious, others include lindane, Aldrin, Dieldrin and chlordane. These are all chemicals noted for their long-lasting effect. i.e. persistence in the environment.
Organophosphates, of which Malathion and Parathion are notable examples, are chemicals designed to be absorbed into the plant from where they are poisonous to insects (mainly mites and aphids) that suck on the plant sap.
Carbamates, such as Carbamyl and Carbofuran are chemicals noted for their rapid elimination from human and animal tissue despite which their toxicity has resulted in them being banned in many countries. The carbamate mode of action and toxicity is similar to the organophosphates which is nerve paralysis due to inhibition of the enzyme cholinesterase.

It will be appreciated that despite the immense economic benefits and increased crop yields that have been derived from agricultural insecticides the cost in terms of long-term toxicity to humans and animals and the ecological damage due to persistence in the environment and loss of essential pollinating insects has been, and remains, a matter of grave concern for humanity.

The range of insecticidal products that are available for human and animal healthcare is less extensive. In human healthcare the available insecticides are currently based on synthetic derivatives of Pyrethrum (a natural extract of Chrysanthemum flowers) which has been widely used in agriculture. Synthetic pyrethroids include Permethrin, Deltamethrin and Cypermethrin all of which are commonly incorporated into products to treat head and body lice, fleas and mite infestation in humans as well as insecticidal sprays for domestic use against flies, beetles, and cockroaches. Synthetic Pyrethroids are known to be toxic, they act on voltage gated sodium channels in nerves causing an influx of sodium and permanent depolarisation. Although commonly used and assumed to be safe when used sparingly there is a considerable body of scientific evidence to suggest more investigation is needed: Holynska-Iwan et. al. Pyrethroids: How they affect Human Health (Medicina 2020, 56, 582).

While insecticides for human use are restricted to those that are thought to be the least toxic, the same restraint is not imposed on products designed for domestic pets. In addition to Pyrethroids two neurotoxic insecticides, Fipronil and Nicotinoid Imidacloprid are commonly found in products to prevent fleas in domestic pets (dogs and cats). Because of the well documented deleterious effect on pollinators Neo-nicotinoids were banned in the US and in Europe but certain exemptions have been allowed. The assumption is that because of the small-scale use in pets these products have little environmental impact. In fact, researchers in the UK have documented levels of these two neurotoxic insecticides in rivers and public waterways in the UK which are far greater than the approved limits: R. Perkins et al: Potential Role of Veterinary Flea Products in Widespread Pesticide Contamination of English Rivers (Science of the Total Environment, Vol 755, part 1, 2021).

Formulations to counteract arthropod borne disease in humans are almost exclusively based on repellents designed to be applied to skin and clothing which are intended to prevent insect bites on exposed areas treated with the repellent, examples include the following:
The synthetic chemical DEET (diethyltoluamide) is considered to be the most effective repellent and is widely used in skin sprays, lotions and even sunscreens to prevent insect bites, it causes discolouration of clothing.
Picaridin is a chemical derived from peppers which has broad spectrum effects combined with low toxicity, it is claimed that it does not damage clothing.
BioUD is an extract of wild tomato consisting primarily of 2-undecanone it is promoted as an effective alternative to DEET
IR3535 is a synthetic chemical (Ethyl Butylacetylaminopropionate) which is reported to offer similar repellent effects as DEET against a wide range of hematophagous insects.
Oil of Lemon Eucalyptus is a natural extract which is promoted as an insect repellent and analgesic it is reported to be effective against a wide range of hematophagous insects.

While all of the above are effective at repelling arthropods and so limiting their bites, none are 100% effective and all depend on availability, diligent skin cover and frequent replenishment during periods of potential exposure. Critically however, none of the readily available insect repellents do anything to inhibit the transfer of pathogens or to ameliorate the effect of an insect bite when repellents fail.

Toxicity and long-term efficacy of many of the commonly used repellents is a significant concern. Permethrin for example is a synthetic pyrethroid which together with similar neurotoxic Insecticidal agents (deltamethrin and Cypermethrin) is used to treat human head lice and scabies, it is known to cause skin rash and irritation and is classified by the US EPA as a carcinogen. Efficacy of all of the pyrethroids is being reduced by a phenomenon known as ‘knockdown resistance’ in insects whereby insects become resistant to these neurotoxic agents.

Currently, human and companion animal protection against hematophagous insects and the infectious disease they can potentially transmit is limited to formulations which repel the insect or in some limited applications kill the insect without addressing the effect of the bite itself and or the infection in the bite.

Furthermore, in the case of tick bites where the insect remains embedded in mammalian skin, conventional intervention involves mechanical intervention with a tweezers or similar tool to pull the insect out of the bite site, a procedure which frequently squeezes the body of the insect causing it to inject the contents of its gut and blood sac into the host, greatly exacerbating the potential for infection. In some cases, mechanical intervention only removes the protruding body parts of the insect, leaving the head embedded and again further exacerbating the potential for infection and host allergic reaction.

An alternative solution to commercially available insecticides, arachnicides and pesticides is needed.

### SUMMARY OF THE INVENTION

In one embodiment, the present invention is a composition comprising an emulsion or a reactive emulsion which comprises a) an effective amount of one or more saturated or unsaturated free fatty acids having from 4 to 22 carbon atoms or an acceptable salt or ester thereof; b) one or more membrane lipids or a hydrolysed derivative thereof, as an emulsifying agent for the free fatty acid(s), or the salt or ester thereof; and c) a diluent or carrier; wherein the fatty acid and membrane lipid together form droplets suspended within the composition, wherein the mean droplet size is less than 1 micron.

In another embodiment the present invention is an insecticidal and/or arachnidicidal composition comprising a) an effective amount of one or more saturated or unsaturated free fatty acids having from 4 to 22 carbon atoms or a pharmaceutically acceptable salt or ester thereof; and one or more membrane lipids or a hydrolysed derivative thereof, as emulsifying agent for the free fatty acid(s) or the salt or ester thereof; and b) a diluent or carrier.

In yet another embodiment, the present invention is a method for the control of insects and/or arachnids, the method comprising contacting said insects and/or arachnids, or a locus at which control is desired, with an effective amount of a composition as described herein.

In yet another embodiment the present invention is a method of treating and/or preventing a physiological and/or an immunological response in a subject in need thereof, wherein said physiological and/or an immunological response is caused by a bite or a sting from an insect and/or arachnid, the method comprising contacting said insects and/or arachnids, said subject, or a locus at which treatment and/or prevention is desired, with a therapeutically effective amount of a composition as described herein.

### DETAILED DESCRIPTION

Surprisingly, it been discovered that the compositions and emulsions of the present invention have unexpected antiparasitic, insecticidal, and/or arachnidicidal effects.

As used herein, an “Insect” is a class of very small animal with six legs, a body divided into three parts and optionally two pairs of wings including, among others, flea, midge, mosquito, horse-fly, tsetse fly, sandfly, blackfly, bedbug, assassin bug, gnat, louse, bee, wasp, hornet, sawfly, ant, caterpillar, aphid, locust, weevil, beetle, whitefly, thrip, scale, earwig, capsid, or mealybug. An insecticide as used herein is a compound or composition useful for the control of insects

As used herein, an “Arachnid” is a class of joint-legged invertebrate animals (arthropods), including, among others, spider, scorpions, tick, mite, pseudoscorpion, harvestmen, camel spider, whip spider or vinegarroon. An Arachnicide as used herein is a compound or composition useful for the control of arachnids.

As used herein, the term “Acari” is a subclass of Arachnid which includes ticks and mites. As used herein an Acaricide is a compound or composition useful for the control of acari.

As used here a “Parasite” is an organism that lives in or on an organism of another species (its host) and/or benefits by deriving nutrients at the host's expense. There are three main classes of parasites that can cause disease in animals: protozoa, helminths, and ectoparasites. As used herein an Antiparasitic is a compound or composition useful for the control of parasites.

As used herein, a “Mollusk” is any of a large phylum of invertebrate animals (as snails, clams, and octopuses) with a soft body lacking segments and usually enclosed in a shell.

As used herein, the term “Control” includes repelling, disabling, immobilizing, incapacitating, paralyzing, crippling, modifying behaviour, rendering moribund, and/or killing, for example a mollusk, parasite, insect and/or arachnid.

As used herein, the term “Pathogen” includes viruses, bacteria, fungi, and parasites (including protozoa, helminths, and ectoparasites).

As used herein, the term “Microbe” is a microorganism of microscopic size, which may exist in its single-celled form or as a colony of cells examples include but are not limited to bacteria, yeast, fungi and viruses. Many pathogens are microbes. As used herein an Antimicrobial is a compound or composition useful for the control of microbes.

In one embodiment, the present invention is an antimicrobial, antiparasitic, insecticidal and/or arachnidicidal composition comprising an emulsion or a reactive emulsion as described herein, and optionally further comprising a diluent or carrier.

In one embodiment, the present invention is an insecticidal and/or arachnidicidal composition comprising an emulsion or a reactive emulsion as described herein, and optionally further comprising a diluent or carrier.

As used herein the terms “antimicrobial, antiparasitic, Insecticidal and/or Arachnidicidal composition” and “Composition” are used interchangeably and refer to the compositions of the present invention comprising emulsions or reactive emulsions of the present invention as described herein, and optionally further comprising a diluent or carrier.

In one embodiment, the present invention is a composition for use in the control of microbes, parasites, mollusks, insects, and/or arachnids.

In one embodiment, the present invention is a composition for use in the control of insects, and/or arachnids. In one embodiment, the present invention is a composition for use in the control of microbes, parasites, mollusks, insects, and/or arachnids at a locus.

In one embodiment, the present invention is a composition for use in the control of mollusks, insects, and/or arachnids at a locus.

In one embodiment, the present invention is a composition for use in the control of microbes, parasites, mollusks, insects, and/or arachnids at a locus, wherein the locus is a plant or a subject.

In one embodiment, the present invention is a composition for use in the control of mollusks, insects, and/or arachnids at a locus, wherein the locus is a plant or a subject.

In one embodiment, the present invention is a composition for use in the control of insects, and/or arachnids.

In one embodiment, the present invention is a composition for use in the control of insects, and/or arachnids at a locus.

In one embodiment, the present invention is a composition for use in the control of insects, and/or arachnids at a locus, wherein the locus is a plant or a subject.

In one embodiment, the present invention is a composition for use in the control of insects and/or arachnids on a plant.

In one embodiment of the present invention, the plant is a feed crop (fruit, vegetable or grain), forage crop (grasses), fiber crop (cotton, hemp or flax), oil crop (canola, olive, soybean or corn), ornamental crop (flowers, hedges, trees), or industrial crop (rubber or tobacco).

In one embodiment, the present invention is a composition for use in the control of insects and/or arachnids on a subject.

In one embodiment, the subject is a human or non-human animal. In one embodiment of the present invention, the subject is a mammal. In one embodiment, the mammal is a human or non-human mammal. In one embodiment, the mammal is a human. In one embodiment of the present invention, the non-human animal is a household pet, farm animal or zoo animal. In one embodiment, the non-human animal is a dog, cat, mouse, rat, gerbil, ferret, hamster, bird, horse, pony, donkey, mule, lama, alpaca, emu, sheep, goat, pig, cow, bull, steer, heifer, deer, tiger, cheetah, wolf, monkey, lion, bear, fox, gorilla, or kangaroo. In one embodiment of the present invention, the subject is not a parasite or arthropod. In one embodiment the subject is a household pet, such as, a dog, cat, bird, rat, mouse, ferret, gerbil or hamster. In one embodiment the subject is a farm animal, such as, a horse, pony, donkey, mule, lama, alpaca, emu, pig, bird, cow, bull, steer, heifer, sheep, or goat. In one embodiment the subject is a zoo animal, such as, bird, deer, tiger, cheetah, wolf, monkey, lion, bear, fox, gorilla, or kangaroo.

In one embodiment of the present invention, the compositions of the invention control an insect and/or arachnid upon contact. Without wishing to be bound by theory, it is believed that on contact with an insect, and/or arachnid, the compositions of the present invention rapidly penetrate the natural orifices including the mouth, anus and/or spiracles, physically blocking the insect and/or arachnid's essential physiological processes and controlling, for example, incapacitating and/or killing the insect and/or arachnid.

As used herein, the term “Contact” includes the insect and/or arachnid attending a locus comprising compositions of the present invention and/or the compositions of the present invention being applied directly to the insect and/or arachnid.

In one embodiment of the present invention, the compositions are contacted with a locus prior to infestation by insects and/or arachnids to prevent insects and/or arachnids infesting said locus. In one embodiment the locus is a subject, such as a human or non-human animal. In one embodiment the locus is a plant.

In another embodiment of the present invention, the compositions are contacted directly with an insect and/or arachnid to treat and/or prevent infestation of a locus by said insects and/or arachnids. In one embodiment the locus is a subject, such as a human or non-human animal. In one embodiment the locus is a plant.

As used herein, the term “Infestation” includes where one or more arthropod, such as, an insect and/or arachnid attends a locus at which their attendance is not desired.

In one embodiment of the present invention, the compositions are contacted with a plant and/or a locus around a plant prior to infestation by insects and/or arachnids to prevent insects and/or arachnids infesting said plant.

In another embodiment of the present invention, the compositions are contacted directly with an insect and/or arachnid to treat and/or prevent infestation of a plant by said insects and/or arachnids.

In one embodiment of the present invention, the compositions are contacted with a plant to treat and/or prevent infestation by insects and/or arachnids by spraying a spray or aerosol or application of a liquid, gel, powder, paste, ointment, cream, surface coating, soap, dry residue, lotion or wipe comprising the compositions of the present invention to and/or around said plant.

In one embodiment of the present invention, the plant, such as, a feed crop (fruit, vegetable or grain), forage crop (grasses), fiber crop (cotton, hemp or flax), oil crop (canola, olive, soybean or corn), ornamental crop (flowers, hedges, trees), or industrial crop (rubber or tobacco) or a locus around the plant is contracted with the compositions of the present invention by spraying a spray or aerosol or application of a liquid, gel, powder, paste, ointment, cream, surface coating, soap, dry residue, lotion or wipe to treat or prevent infestation by insects and/or arachnids.

In one embodiment of the present invention, the compositions are contacted with a subject prior to infestation by insects and/or arachnids to prevent insects and/or arachnids infesting said subject. In one embodiment the subject is a human or non-human animal.

In another embodiment of the present invention, the compositions are contacted directly with an insect and/or arachnid to treat and/or prevent infestation of a subject by said insects and/or arachnids.

In one embodiment of the present invention, the compositions are contacted with a subject to treat or prevent infestation by insects and/or arachnids by spraying a spray or aerosol or application of a liquid, gel, powder, paste, ointment, cream, surface coating, soap, dry residue, lotion or wipe comprising the compositions of the present invention to said subject.

In one embodiment of the present invention, the compositions of the present invention are contacted with a subject, for example, human, dog, cat, mouse, rat, gerbil, ferret, hamster, bird, horse, pony, donkey, mule, lama, alpaca, emu, sheep, goat, pig, cow, bull, steer, heifer, deer, tiger, cheetah, wolf, monkey, lion, bear, fox, gorilla, or kangaroo by spraying a spray or aerosol or application of a liquid, gel, powder, paste, ointment, cream, surface coating, soap, dry residue, lotion or wipe comprising the compositions of the present invention to treat or prevent infestation by insects and/or arachnids.

In one embodiment of the present invention, the compositions of the present invention are contacted with a subject, for example, human, dog, cat, gerbil, ferret, hamster, horse, pony, donkey, mule, sheep, goat, pig, cow, bull, steer or heifer spraying a spray or aerosol or application of a liquid, gel, powder, paste, ointment, cream, surface coating, soap, dry residue, lotion or wipe comprising the compositions of the present invention to treat or prevent infestation by insects and/or arachnids.

In one embodiment of the present invention, the compositions of the present invention modify the insect and/or arachnid behaviour at a locus, for example, a plant or subject, for example, repelling the insect and/or arachnid from the plant or subject thus preventing it from eating said plant or biting said subject and/or causing the insect, and/or arachnid to release itself from said subject after biting and/or prior to completion of its blood meal and thus reducing the transmission of a pathogen to a subject.

In one embodiment of the present invention, the compositions of the present invention modify the biological mechanism that insects and/or arachnids use to anchor in a bite site of a subject. In one embodiment of the present invention, the compositions of the present invention cause insects and/or arachnids to be dislodged or to dislodge themselves from a bite site on a subject. In one embodiment of the present invention, the compositions of the present invention modify the biochemistry of a secretion from an arthropod. In one embodiment, a secretion from an arthropod includes, for example, insects and/or arachnid saliva which comprises, for example, a biochemical cement that, for example, ticks use to anchor in a bite site of a subject.

As used herein, the term “Cement” includes any secretion from an insect and/or arachnid which is or comprises a component would aid in the adhesion of the insect and/or arachnid to a bite site.

In one embodiment of the present invention, the insect and/or arachnid is a flea, midge, mosquito, horsefly, gnat, louse, spider, tick, or mite.

In one embodiment of the present invention, the insect and/or arachnid is a flea, midge, mosquito, horsefly, tick, or mite.

In one embodiment of the present invention, the insect and/or arachnid is a flea, midge, horsefly, tick, or mite.

In one embodiment of the present invention, the arachnid is a tick.

In one embodiment, the invention is a composition useful for control of insects, and/or arachnids, in particular, hematophagous organisms which can act as vectors for a great variety of pathogens, including viruses, bacteria, fungi, and parasites.

In one embodiment, the invention is a composition for use in treating and/or preventing a disease in a subject in need thereof, wherein said disease is caused by a pathogen transmitted to said subject by an insect, arachnid, and/or parasite the method comprising contacting said parasites, insects and/or arachnids, a locus at which treatment and/or prevention is desired, or said subject, with a therapeutically effective amount of a composition of the present invention.

In one embodiment, the invention is a topical composition for use in treating and/or preventing a disease in a subject in need thereof, wherein said disease is caused by a pathogen transmitted to said subject by an insect, arachnid, and/or parasite comprising the method comprising contacting said insects and/or arachnids, a locus at which treatment and/or prevention is desired, or said subject, with a therapeutically effective amount of a topical composition of the present invention. In one embodiment, the invention is a topical composition for use in treating and/or preventing a subdermal, intradermal or subcutaneous disease in a subject, wherein said disease is caused by a pathogen transmitted to said subject by an insect, arachnid, and/or parasite the method comprising contacting said insects and/or arachnids, a locus at which treatment and/or prevention is desired, or said subject, with a therapeutically effective amount of a composition of the present invention.

In one embodiment the disease is Anaplasmosis, Human Granulocytic Anaplasmosis, Babesiosis, Borrelia mayonii infection, Borrelia miyamotoi infection, Bourbon virus infection, Colorado tick fever, Alkhurma Haemorrhagic Fever, Ehrlichiosis, Heartland virus, Lyme disease, Powassan disease, Rickettsia parkeri rickettsiosis, Rocky Mountain spotted fever (RMSF), STARI (Southern tick-associated rash illness), Tickborne relapsing fever (TBRF), Tularemia, Rickettsiosis, Tick borne encephalitis, Akabane virus, Schmallenberg, Blue-tongue, Equine infectious anaemia, Anthrax, Trypanosomes (trypanosoma/Chagas), Malaria, Dengue, Yellow fever, Zika virus, Chikungunya, Human Lymphatic Filariasis, Black death, Plague, Bubonic plaque, Rickettsia disease, Tungiasis, Chiggers, Scabies, Typhus fever, Parasite infestation, Crimean-Congo Haemorrhagic Fever, Rickettsiosis, or Yersinia pestis.

In one embodiment the parasite, insect and/or arachnid is flea, midge, mosquito, horse-fly, tsetse fly, sandfly, blackfly, bedbug, assassin bug, gnat, louse, sawfly, spider, tick, mite, scorpion, pseudoscorpion, harvestmen, camel spider, whip spider, or vinegarroon. In one embodiment the insect is a flea, midge, mosquito, horse-fly, tsetse fly, sandfly, blackfly, bedbug, assassin bug, gnat, louse, sawfly, or ant. In one embodiment the Arachnid is a spider, tick, mite, scorpion, pseudoscorpions, harvestmen or vinegarroons. In one embodiment, the flea is Tunga penetrans, chigoe, chigo, chigoe flea, chigo flea, jigger, nigua, sand flea, or burrowing flea. In one embodiment the mite is Sarcoptes scabiei var. hominis, Trombiculidae; harvest mite, berry bug, bush-mite, red bus or scrub-itch mite. In one embodiment the parasite is Loa loa.

In one embodiment, the invention is a composition for use in treating and/or preventing a disease in a subject in need thereof, wherein said disease is caused by a pathogen transmitted by an hematophagous organism to said subject, comprising contacting said organism, a locus at which treatment and/or prevention is desired, or said subject with a therapeutically effective amount composition of the present invention and a pharmaceutically acceptable diluent or carrier.

In one embodiment the disease is Anaplasmosis, Human Granulocytic Anaplasmosis, Babesiosis, Borrelia mayonii infection, Borrelia miyamotoi infection, Bourbon virus infection, Colorado tick fever, Alkhurma Haemorrhagic Fever, Ehrlichiosis, Heartland virus, Lyme disease, Powassan disease, Rickettsia parkeri rickettsiosis, Rocky Mountain spotted fever (RMSF), STARI (Southern tick-associated rash illness), Tickborne relapsing fever (TBRF), Tularemia, Rickettsiosis, Tick borne encephalitis, Akabane virus, Schmallenberg, Blue-tongue, Equine infectious anaemia, Anthrax, Trypanosomes (trypanosoma/Chagas), Malaria, Dengue, Yellow fever, Zika virus, Chikungunya, Human Lymphatic Filariasis, Black death, Plague, Bubonic plaque, Rickettsia disease, Tungiasis, Chiggers, Scabies, Typhus fever, Parasite infestation, Crimean-Congo Haemorrhagic Fever, Rickettsiosis, or Yersinia pestis.

In one embodiment the parasite, insect and/or arachnid is flea, midge, mosquito, horse-fly, tsetse fly, sandfly, blackfly, bedbug, assassin bug, gnat, louse, sawfly, ant, spider, tick, mite, scorpion, pseudoscorpion, harvestmen, camel spider, whip spider, or vinegarroon. In one embodiment the insect is a flea, midge, mosquito, horse-fly, tsetse fly, sandfly, blackfly, bedbug, assassin bug, gnat, louse, sawfly, or ant. In one embodiment the Arachnid is a spider, tick, mite, scorpion, pseudoscorpions, harvestmen or vinegarroons. In one embodiment, the flea is Tunga penetrans, chigoe, chigo, chigoe flea, chigo flea, jigger, nigua, sand flea, or burrowing flea. In one embodiment the mite is Sarcoptes scabiei var. hominis, Trombiculidae; harvest mite, berry bug, bush-mite, red bus or scrub-itch mite. In one embodiment the parasite is Loa loa. In one embodiment the compositions of the present invention treat and/or prevent a subdermal, intradermal or subcutaneous disease in a subject.

In one embodiment the compositions of the present invention are applied topically to a subject and treat and/or prevent a subdermal, intradermal or subcutaneous disease in said subject.

In one embodiment, the invention is a composition for use in controlling a parasite, insect and/or arachnid and treating and/or preventing a disease in a subject transmitted to said subject by said parasite, insect and/or arachnid.

In one embodiment, the invention is an insecticidal, arachnicidal, antiparasitic and/or antimicrobial composition comprising an emulsion or a reactive emulsion of the present invention as described herein.

In one embodiment of the present invention, the compositions of the present invention penetrates the skin of a subject and controls a pathogen, such as a parasite that may be embedded there (e.g., Trombiculidae, Sarcoptes scabiei var. hominis, Tunga penetrans) and treats and/or prevents the disease caused by said parasite (e.g., chiggers, scabies, and/or tungiasis).

In one embodiment, the invention is a composition for use in suppressing, treating and/or preventing a subject's physiological and/or immunological responses to a bite or a sting from an insect and/or arachnid.

In one embodiment, the invention is a composition for use in suppressing, treating and/or preventing a subject's physiological and/or immunological responses to a bite from a hematophagous organism.

In one embodiment of the present invention, the compositions of the present invention suppress, treat and/or prevent a subject's physiological and/or immunological response to an insect and/or arachnid secretion at a bite or sting site to reduce, for example, inflammation; itchiness; erythema; hives; redness; pain; vasoconstriction; platelet aggregation; degranulation of mast cells; release of dendritic cells; activation of fibroblasts, fibrinogen, bradykinins, histamines, or chemoattractant (chemokines or leukotrienes); transport of neutrophils or monocytes; or generating antigen specific antibodies or T lymphocytes.

In another embodiment of the present invention, the compositions of the present invention suppress, treat and/or prevent a subject's physiological and/or immunological response to a secretion of, for example, a flea, midge, mosquito, horse-fly, tsetse fly, sandfly, blackfly, bedbug, assassin bug, gnat, louse, sawfly, ant, spider, tick, mite, scorpion, pseudoscorpion, harvestmen, camel spider, whip spider, or vinegarroon at a bite/sting site.

In another embodiment of the present invention, the compositions of the present invention suppress, treat and/or prevent a subject's physiological and/or immunological response to a secretion of, for example, a flea, midge, mosquito, horse-fly, bedbug, gnat, louse, spider, tick, or mite at a bite/sting site.

In another embodiment of the present invention, the compositions of the present invention suppress, treat and/or prevent a subject's physiological and/or immunological response to a secretion of, for example, a flea, midge, mosquito, horse-fly, gnat, louse, tick, or mite at a bite site.

In another embodiment of the present invention, the compositions of the present invention suppress, treat and/or prevent a subject's physiological and/or immunological response to a secretion of, for example, a flea, midge, mosquito, horse-fly tick, or mite at a bite site.

In another embodiment of the present invention, the compositions of the present invention suppress, treat and/or prevent a subject's physiological and/or immunological response to a secretion of a tick or mite at a bite site.

In another embodiment of the present invention, the compositions of the present invention suppress, treat and/or prevent a subject's physiological and/or immunological response to a secretion of a tick a bite site.

In another embodiment of the present invention, the compositions of the present invention suppress, treat and/or prevent a subject's physiological and/or immunological response to a secretion of a mosquito at a bite site.

It has also unexpectedly been discovered that the emulsions and reactive emulsions of the present invention can be used to deliver oil soluble active ingredients, such as for example, insecticides. In one embodiment of the present invention, the emulsions or reactive emulsions of the present further comprise an active ingredient. In one embodiment of the present invention, the emulsions or reactive emulsions of the present further comprise an antimicrobial, antiparasitic, insecticidal and/or arachnidicidal composition. In one embodiment of the present invention, the emulsions or reactive emulsions of the present further comprise an insecticidal composition. This embodiment allows for safer delivery of active ingredients including, for example, insecticides at much lower doses where potency is amplified by delivery in the emulsion or reactive emulsion of the present invention.

In another embodiment of the present invention, the emulsions and reactive emulsions further comprise a wetting agent in the water phase. In one embodiment, the insecticidal potency of the emulsions and reactive emulsions of the present invention is amplified by the addition of a wetting agent. In one embodiment of the present invention, potency of the emulsions and reactive emulsions is increased by about 10% with the addition of a wetting agent. Suitable wetting agents include glycerol, polysorbate (Tween), propylene glycol and sodium lauryl sulphate. In one embodiment, the emulsions and reactive emulsions of the present invention further comprise a wetting agent which comprises between about 0.1% and about 10%, between about 0.5% and about 5%, or between about 1% and about 5% w/w of the water phase.

In another embodiment of the present invention, the emulsions and reactive emulsions further comprise an amino acid derivative. Suitable amino acid derivative include N-Acetyl Cysteine and/or Pyrrolidone Carboxylic Acid/Pyroglutamic Acid. In one embodiment, the emulsions and reactive emulsions of the present invention further comprise an amino acid derivative at between about 0.1% and about 10%, between about 0.5% and about 5%, or between about 1% and about 2% w/w of the water phase. In one embodiment of the present invention, the emulsions and reactive emulsions further comprise N-Acetyl Cysteine and/or Pyrrolidone Carboxylic Acid/Pyroglutamic Acid. In one embodiment the compositions of the present invention are non-toxic to a subject.

As used herein, the term “Non-toxic compositions” includes compositions of the present invention which when contacted with a subject at the doses specified herein are not known to cause any significant adverse effect in said subject. As used herein, the phrase “side effects” encompasses unwanted and adverse effects. Side effects are always unwanted, but unwanted effects are not necessarily adverse. An adverse effect from a therapy (e.g., prophylactic or therapeutic) might be uncomfortable, harmful, or lethal. Side effects include, but are not limited to fever, chills, lethargy, immunological reactions, gastrointestinal toxicities (including gastric and intestinal ulcerations and erosions), nausea, vomiting, neurotoxicities, nephrotoxicities, renal toxicities (including such conditions as papillary necrosis and chronic interstitial nephritis), hepatic toxicities (including elevated serum liver enzyme levels), myelotoxicities (including leukopenia, myelosuppression, thrombocytopenia and anaemia), dry mouth, metallic taste, prolongation of gestation, weakness, somnolence, pain (including muscle pain, bone pain and headache), hair loss, asthenia, dizziness, extra-pyramidal symptoms, akathisia, cardiovascular disturbances and sexual dysfunction.

In one embodiment, the compositions of the present invention are biodegradable.

In one embodiment, the compositions of the present invention not persistent in the environment.

In one embodiment, the compositions of the present invention do not affect pollinating insects

In one embodiment, the compositions of the present invention do not present a toxic residue which might enter the human food chain.

In one embodiment the compositions of the present invention are significantly safer than commercially available insecticides and pesticides. The emulsions of this invention have been shown to exert minimal cytotoxicity in a wide range of cell types, they have been shown to be safe in topical and oral application, by acute I. V. administration and nasal instillation. The components of these emulsions are commonly found in human foods and are safe if accidentally ingested. (Purves et. Al. J. Gen Virol 2023; 104:001821)

In one embodiment, the emulsions used in the compositions of the present invention are based on components normally found in food, and frequently used in human parenteral nutrition. As such they are infinitely safer than any of the existing chemical interventions used to protect plant, animal and human against arthropod assault.

In one embodiment, the present invention is composition comprising an emulsion or a reactive emulsion of free fatty acids in de-lipidised membranes, wherein the fatty acid (oil) is surrounded by the de-lipidised membrane amphipath) in droplet form, wherein the mean droplet size is less than 1 micron, preferably in the region of between 0.8 and 0.6 micron, and preferably less than 0.5 micron.

As used herein the emulsion “Droplet” size refers to the size of a droplet comprising oil (fatty acid and optionally, for example, triglyceride) surrounded by/coated with de-lipidised membrane, wherein the droplet is suspended in the aqueous layer (for example, diluent carrier) of the compositions of the present invention. In one embodiment, the compositions of the present invention comprise emulsions of free fatty acids in de-lipidised membranes.

Emulsions of free fatty acids in de-lipidised lecithin useful in the compositions of the present invention are described in WO 2011 061237 the entire contents of which is incorporated here by reference. The manufacturing methods of the emulsions described in WO 2011 061237 can be used in the preparation of the compositions of the present invention. In addition to the methods described in WO 2011 061237, a secondary high pressure homogenisation step has been shown to greatly enhance the insecticidal and arachnidicidal activity of the emulsions used in the compositions of the present invention. The enhanced manufacturing method is described in the methods section herein. Further studies of properties of the emulsions useful in the compositions of the present invention are reported in Fletcher et. Al. A Novel Antiviral Formulation Inhibits a Range of Enveloped Viruses (J. General Virology 2020; 101: pp 1090-1102), the entire contents of which is incorporated herein by reference.

In one embodiment, the emulsions of the present invention are formed using homogenisation/micronization by, for example, extrusion or agitation of the lipids and fatty acids

In one embodiment, the present invention is a method of manufacturing the emulsions of the present invention, the method comprising:
suspending a de-lipidised amphipath in an aqueous medium (for example, diluent or carrier)
adding an oil, under mechanical agitation, to form an emulsion or a reactive emulsion comprising droplets of the oil and amphipath, and
homogenizing said emulsion by, for example, vigorous agitation to reduce droplet size to optimize, for example, efficacy and stability of the emulsion.

In one embodiment, the homogenized emulsions of the present invention with a mean droplet size of less than 1 micron show enhanced efficacy and stability versus non-homogenised emulsions.

As used herein, an “Emulsion” is a dispersion of one immiscible liquid in an immiscible matrix. Typically emulsions are either oil in water or water in oil dispersions wherein the dispersion is prevented from coalescing by an emulsification agent which is usually amphipathic. The term emulsion includes an oil in water or water in oil dispersion stabilised with amphipathic molecules which may be formed remotely prior to administration or In Situ by administering a composition of precursors which assemble into an emulsion at the intended site of action. The emulsions useful in the compositions of the present invention are based on one or a combination of oils, such as, free fatty acids selected from Caproic, Caprylic, Capric, Lauric, Undecylenic, Myristic, Palmitic, palmitoleic, Oleic, Stearic, lineic, linoleic, or linolenic, preferably Caprylic, Capric, undecylenic, lauric and Oleic and more preferably caprylic and Capric. The emulsions of the present invention comprise an oil phase and a water phase. In one embodiment, the emulsions of the present invention are reactive emulsions.

As used herein, a “reactive emulsion” is an emulsion with a depletable oil phase caused by the conversion of the free fatty acids, contained in the reactive emulsion, from oil soluble free acids to water soluble salts (soaps) when the reactive emulsion is subjected to pH change. This transition creates an amphipath with open lipophilic sites in the reactive emulsions. Having an amphipath with open lipophilic sites at a target site allows the amphipath to associate with other lipophilic surfaces such as adjacent cell membranes or an insect cuticle. In addition, when a lipophilic surface is coated with an amphipath it causes a phase inversion on that surface, inverting it from lipophilic to hydrophilic—i.e. water repelling to water absorbing. This inversion facilitates migration of water, and/or intact emulsion droplets, into insect spiracles and other body orifices. In one embodiment, the reactive emulsions of the present invention create a surface phase inversion at a desired surface, allowing droplets to migrate along said inverted surface. A selection of insects have a raised ‘lip’ around the spiracle. Where the surface of the lip remains lipophilic this lip is an impediment to aqueous phase ingress. In one embodiment of the present invention, the reactive emulsions of the present invention cause surface phase inversion of a lipophilic surface causing the water repelling effect to be neutralised and/or replaced with phase attraction, thus the instant emulsion droplets can migrate over the surface, for example, the lip and into the spiracle. The terms “reactive emulsion” and “emulsion” can be used herein interchangeably. In one embodiment of the present invention, the presence of a free fatty acid in the emulsion facilitates phase inversion and insecticidal effect of the emulsions and reactive emulsions of the present invention. The use of a free fatty acid as a depletable oil phase facilitates reactivity and delivery of an amphipath with exposed lipophilic sites and consequential surface phase inversion. The ratio of free fatty acid to triglyceride can be used to control rate of reactivity. In one embodiment, the oil phase of the emulsions and reactive emulsions of the present invention comprises at least 50% the free fatty acid. In one embodiment, the oil phase of the emulsions or reactive emulsions of the present invention comprise between about 10 and about 90% fatty acid, between about 10% and about 70% fatty acid, between about 10% and about 60% fatty acid, between about 10% and about 50% fatty acid, between about 20% and about 50% fatty acid, between about 30% and about 50% fatty acid, or between about 40% and about 50% fatty acid. Reduced efficacy is detectable at inclusion rates down to 10% but there is no detectable efficacy if the oil phase is 100% triglyceride and no free fatty acid.

Free fatty acids behave as water insoluble oils at pH values below their dissociation constant, which is normally in the region of pH 5.5. When neutralised at pH above their dissociation constant, free fatty acids become water soluble salts, and this conversion from water insoluble oil to water soluble salt is used in the emulsions of this invention to facilitate delivery of a lipophilic amphipath to a required site of action.

When protonated as free acids, fatty acids are oils which may be freely dispersed in other oils and triglycerides. Triglycerides are fats/oils comprising a glycerol back-bone with three fatty acids esterified to each of the three hydroxyl groups of glycerol. Typically triglycerides have three different esterified fatty acids. Manipulation of the fatty acid composition of a triglyceride is achieved by chemical synthesis and so triglycerides consisting exclusively of caprylic acid are available commercially as are mixed capric/caprylic triglycerides which behave as light neutral oils.

Dispersion of a free fatty acid or combination thereof in triglyceride oils permits manipulation of the availability of free fatty acid and its conversion rate from free acid oil to water soluble salt which in turn affects rate of availability of de-lipidised amphipath and the anti-arthropod performance characteristics of the emulsion as described in this invention.

In one embodiment, the compositions of the present invention comprise an emulsion or a reactive emulsion which comprises one or more saturated or unsaturated free fatty acids having from 4 to 22 carbon atoms or a pharmaceutically acceptable salt or ester thereof; and one or more membrane lipids or a hydrolysed derivative thereof, as emulsifying agent for the free fatty acid(s) or the salt or ester thereof; and the composition further comprises a diluent or carrier.

In one embodiment, the free fatty acid is selected from valeric, caproic, caprylic, pelargonic, capric, undecanoic, undecylenic, lauric, myristic, palmitic, stearic, oleic, linoleic and linolenic acids and mixtures thereof, and pharmaceutically acceptable salts and esters thereof. In one embodiment, the free fatty acid is selected from one or more of caproic, caprylic, pelargonic, capric, undecylenic and lauric acids, especially caprylic acid. In one embodiment, the free fatty acid is selected from caprylic and capric acids. In one embodiment, wherein the free fatty acid is caprylic acid.

In one embodiment, the membrane lipid is selected from one or more of phospholipids, lecithin, glycerophospholipids, sphingolipids, glycosphingolipids, glycoglycerolipids and cholesterols, and hydrolysed derivatives thereof. In one embodiment, the membrane lipid is selected from one or more of phosphatidic acid, phosphatidylcholine, phosphatidylethanolamine, phosphatidylglycerol, phosphatidylinositol, phosphatidylserine, lecithin, ceramide, sphingomyelin, glycolipids, glycosphingolipids, cerebrosides, gangliosides, glycoglycerolipids, mono-galactosyl diglyceride, lanosterol and cholesterol. In one embodiment, the membrane lipid is a phospholipid selected from one or more of phosphatidic acid, phosphatidylcholine, phosphatidylethanolamine, phosphatidylglycerol, phosphatidylinositol, phosphatidylserine and lecithin, especially lecithin.

In one embodiment, the membrane lipid or hydrolysed derivative thereof is de-lipidised. In one embodiment, the membrane lipid or hydrolysed derivative thereof is de-lipidised lecithin.

In one embodiment, the de-lipidised lecithin contains less than 3% conjugated extraneous lipid material.

In one embodiment, the compositions of the present invention comprise an emulsion or a reactive emulsion which comprises a free fatty acid selected from one or more of caproic, caprylic, pelargonic, capric, undecylenic and lauric acids, in combination with de-lipidised lecithin.

In one embodiment, the compositions of the present invention comprise an emulsion or a reactive emulsion which comprises a weight ratio of fatty acids to membrane lipids is from about 0.25:1 to about 10:1; or from about 0.5:1 to about 10:1, or from about 0.5:1 to about 5.0:1, or from about 1.0:1 to about 2.5:1, or from about 1.25:1 to about 2.5:1, on a weight for weight basis.

In one embodiment, the compositions of the present invention comprise an emulsion or a reactive emulsion which comprises (a) one or more free fatty acids selected from the group consisting of caproic, caprylic, capric, lauric, palmitic, stearic, oleic, linoleic and linolenic acids and mixtures thereof; and (b) one or more membrane lipids as emulsifying agent for the free fatty acid(s), wherein the membrane lipid is delipidized lecithin, wherein the weight ratio of component (a) to component (b) is from about 0.25:1 to about 10:1.

In one embodiment, the compositions of the present invention further comprise one or more pharmaceutically acceptable organic acids or a pharmaceutically acceptable salt or ester thereof; and/or one or more pharmaceutically acceptable organic acid salts.

In one embodiment, the organic acid is selected from acetic, pyruvic, propionic, glycolic, oxalic, lactic, glyceric, tartronic, malic, maleic, ascorbic, fumaric, tartaric, malonic, glutaric, propenoic, cis or trans butanoic and citric acids and mixtures thereof, and pharmaceutically acceptable salts and esters thereof. In one embodiment, the organic acid is citric or lactic acid or the sodium or potassium salt thereof. In one embodiment, the organic acid salt is sodium citrate.

In one embodiment, the organic acid salt is selected from sodium, potassium and calcium

In one embodiment the organic acid is a fatty acid salt which may be combined with de-lipidised lecithin formulated as a powder or paste which may be subsequently hydrated with an acidic solution causing the fatty acid salt to transition to a free acid oil spontaneously forming and emulsion with the de-lipidised lecithin as described in WO2021/150501 the entire contents of which are incorporated herein by reference.

In one embodiment, the emulsion is an oil-in-water emulsion.

In one embodiment, the composition is formulated as a spray, aerosol, liquid, gel, powder, paste, ointment, cream, surface coating, soap, dry residue, lotion, or wipe.

Amphipathic molecules are characterised as macromolecules with one aspect being lipophilic (oil soluble) and the opposing aspect being hydrophilic (water soluble). Such molecules are typically found in nature as membrane molecules interfacing between oil/lipid rich environments and largely aqueous environments. An example of a naturally occurring amphipathic molecules are the fat globule membranes that stabilise butterfat droplets dispersed in the aqueous protein rich matrix of milk.

In a natural environment amphipaths are normally closely associated or conjugated to lipid or lipid like material, as such their lipophilic sites are fully occupied. In order to make use of natural amphipaths in the emulsions of this invention it is first necessary to remove all externally conjugated lipid, this is achieved by suspension and extraction into a solvent such as acetone.

Once fully de-lipidised the amphipath can be suspended in an aqueous medium and when an oil is added under vigorous agitation an emulsion or a reactive emulsion will be formed with oil droplets surrounded by the amphipath, its lipophilic sites orientated into the oil and its opposing hydrophilic sites oriented into the water phase. And it should be noted that in this emulsified state the lipophilic sites of the de-lipidised amphipath are again fully occupied by the oil phase.

Almost any oil or fat can be emulsified in a de-lipidised amphipath. Oils used in the product of this invention however must contain at least one or more free fatty acids, preferably short to medium chain saturated fatty acids and optionally these may be dispersed in a triglyceride wherein the triglyceride is no more that 60% by weight of the total oil (oil phase).

In one embodiment, suitable amphipaths may be selected from lanolin, cholesterol, ceramide or lecithin, preferably lecithin may be used in the composition of the present invention. Lecithin is a composition of five separate phospholipids (phosphatidyl choline, phosphatidyl ethanolamine, phosphatidyl serine, phosphatidyl inositol and phosphatidic acid), which may exist in different ratios depending on the source of the lecithin (soy or egg yolk are common). Lecithin containing no less than 60% phosphatidyl choline and no more that 5% phosphatidic acid are preferred for this invention.

It is an object of this invention to present an amphipath with its lipophilic sites exposed and free to associate with targeted lipophilic surfaces such as the external surfaces of enveloped viruses, bacteria and mammalian epithelia and including as disclosed herein the waxy surfaces of arthropods. Delivery of a free lipophilic amphipath is achieved by using a depletable oil phase—an oil which will provisionally block the amphipath's lipophilic sites and subsequently free them when required by transitioning from oil to a water-soluble salt.

While not wishing to be bound by theory, the surfactant and adhesion inhibitory properties of free fatty acid emulsions is thought to be due to the fact that free fatty acids will transition from oils to water soluble salts when subjected to a pH shift above their pKa and in doing so they expose lipophilic sites on the emulsified amphipath which are free to associate with other lipophilic sites in the immediate environment.

As disclosed in this invention reactive emulsions of free fatty acids in de-lipidised amphipath will readily penetrate full thickness animal, such as, porcine skin and will exert an antimicrobial, insecticidal and adhesion inhibitory effect in the subcutaneous tissue. Further, as demonstrated here topical application of free fatty acid emulsions of this invention will modify the physiological response to insect bite significantly reducing erythema and the irritation (itchiness) normally associated with an insect bite.

Emulsions of free fatty acids in de-lipidised amphipaths as disclosed in this invention will degrade to mixtures of salts of fatty acids and de-lipidised amphipath when subjected to physiological pH values above the pKa of the fatty acid. The normal pH of mammalian skin is 5.5 but subdermal pH tends towards neutrality. Free fatty acid emulsions on a skin surface will remain relatively stable but when these emulsions migrate through the skin (at an insect bite site for example) they will encounter pH values which will deplete the oil phase, forming water soluble salts of free fatty acids and amphipaths with exposed lipophilic sites. These exposed lipophilic sites will readily bind to other lipophilic surfaces and/or serve as a binding site for lipophilic macromolecules in the vicinity.

Most mammalian cells have lipophilic surface properties and lipophilic phase attraction is the primary physio-chemical driver for initial association between cells, including association of potential pathogens to mammalian cell surfaces and the association of biochemical ligands to specific receptor sites.

In addition to topical insect repellent and insecticidal effect, the present invention is a method for subdermal, intradermal, or subcutaneous suppression inhibition of a physiological and/or immunological response to insect and/or arachnid bite or sting and subdermal, intradermal, or subcutaneous suppression inhibition of the transmission of arthropod borne infectious disease.

Subdermal amphipaths with exposed lipophilic sites as may be generated In Situ by transdermal absorption of micro-emulsions of this invention will transiently bind to lipophilic cell surfaces and in so doing will render these cells less accessible to interaction with adjacent cells and with agonist or antagonistic macromolecules in insect saliva and/or pathogens that may be contained therein.

Transdermal presentation of a free fatty acid emulsion which degrades to a composition of salts of free fatty acids and amphipathic molecules with exposed lipophilic sites will generate significant non-specific perturbation of the physiology and biochemical interactivity in the immediate subcutaneous environment. It should be noted that salts of free fatty acids are in fact soaps and exert a significant surfactant effect which may be considered to act independently of and in consort with the lipophilic amphipaths to transiently blind cell surface receptor sites and disrupt the biological activity of components of insect and/or arachnid secretion e.g., saliva, such as:
Salivary inhibitors of vasoconstriction will be blocked or de-activated;
Anti-coagulation factors will be inactivated or overcome by pro-coagulation properties of phospholipid amphipaths which mimic platelet activating factors;
De-granulation of mast cells will be inhibited;
Histamine receptors will be blocked;
Acquisition of infectious agents by dendritic cells and transport from the bite site will be inhibited; and/or
Pathogens will be inactivated and killed before they migrate from the bite site.

In one embodiment of the present invention, the composition is formulated as an emulsion or a reactive emulsion, as described herein, optionally in concentrate form, incorporated and diluted in a carrier formulated as a spray, aerosol, liquid, gel, powder, paste, ointment, cream, surface coating, soap, dry residue, lotion or wipe which exerts an insecticidal and/or arachnicidal effect, and which by virtue of its surfactant nature will rapidly migrate into an insect and/or arachnid bite/sting site inhibiting the infectivity of arthropod borne pathogens; modifying a subject's physiological and/or immunological response to secretions by said insect and/or arachnid; and/or controlling said insect and/or arachnid.

In one embodiment, the present invention is a method for the control of insects and/or arachnids the method comprising contacting said insects and/or arachnids, or a locus at which control is desired, with an effective amount of a composition of the present invention.

In one embodiment, the present invention is a method for the control of insects and/or arachnids at a locus the method comprising contacting said insects and/or arachnids, or said locus, with an effective amount of a composition of the present invention.

In one embodiment, the locus is a plant including the locus on or around the plant.

In one embodiment of the present invention, the plant is a feed crop (e.g., fruit, vegetable or grain), forage crop (e.g., grasses), fiber crop (e.g., cotton, hemp or flax), oil crop (e.g., canola, olive, soybean or corn), ornamental crop (e.g., flowers, hedges, trees), or industrial crop (e.g., rubber or tobacco).

In one embodiment, the present invention is a method for control of insects, and/or arachnids on a plant the method comprising contacting said plant and/or the locus around said plant, with an effective amount of a composition of the present invention.

In one embodiment, the present invention is a method for control of insects, and/or arachnids on a plant the method comprising contacting said plant and/or the locus around said plant, with an effective amount of a composition of the present invention.

In one embodiment, the present invention is a method for control of insects, and/or arachnids on a plant the method comprising contacting said plant and/or the locus around said plant, with an effective amount of a composition of the present invention once, twice or more during the growing season.

In one embodiment, the present invention is a method for control of insects, and/or arachnids on a plant the method comprising contacting said plant and/or the locus around said plant, with an effective amount of a composition of the present invention daily, weekly, biweekly, monthly, or annually.

In one embodiment, the present invention is a method for control of insects, and/or arachnids on a plant the method comprising contacting said plant and/or the locus around said plant, with an effective amount of a composition of the present invention prior to planting the plant.

In one embodiment, the insect and/or arachnid is a caterpillar, aphid, locust, weevil, beetle, whitefly, thrip, scale, earwig, capsid, or mealybug.

In one embodiment the plant, locus or insect and/or arachnid is contacted with the compositions of the present invention by spraying a spray or aerosol or application of a liquid, gel, powder, paste, ointment, cream, surface coating, soap, dry residue, lotion or wipe comprising the compositions of the present invention.

In one embodiment, the locus is a subject including the locus on or around the subject.

In one embodiment, the subject is a human or non-human animal. In one embodiment, the subject is a mammal. In one embodiment, the mammal is a human or non-human mammal. In one embodiment, the non-human animal is a household pet, farm animal, or zoo animal. In one embodiment, the non-human animal is a dog, cat, mouse, rat, gerbil, ferret, hamster, bird, horse, pony, donkey, mule, lama, alpaca, emu, sheep, goat, pig, cow, bull, steer, heifer, deer, tiger, cheetah, wolf, monkey, lion, bear, fox, gorilla, or kangaroo. In one embodiment, the subject is not a parasite or arthropod.

In one embodiment, the present invention is a method for control of insects and/or arachnids on a subject, the method comprising contacting said insects and/or arachnids, a locus at which control is desired, said subject, or an area on or around said subject, such as clothing, blankets, collars, saddles, head collars, tags, jewellery etc., with an effective amount of a composition of the present invention.

In one embodiment, the present invention is a method for control of insects and/or arachnids on a subject, the method comprising contacting said insects and/or arachnids, a locus at which control is desired, or said subject, with an effective amount of a composition of the present invention ad libitum.

In one embodiment, the present invention is a method for control of insects and/or arachnids on a subject, the method comprising contacting said insects and/or arachnids, a locus at which control is desired, or said subject, with an effective amount of a composition of the present invention prior to an encounter with said insects and/or arachnids.

In one embodiment of the methods of the present invention, the present invention is a method for control of insects and/or arachnids on a subject, the method comprising contacting said insects and/or arachnids, a locus at which control is desired, or said subject, with an effective amount of a composition of the present invention daily, weekly, bi-weekly, monthly or annually.

In one embodiment of methods of the present invention, the compositions are contacted with an insect and/or arachnid, locus or subject by spraying a spray or aerosol or application of a liquid, gel, powder, paste, ointment, cream, surface coating, soap, dry residue, lotion or wipe comprising the compositions of the present invention.

In one embodiment of methods of the present invention, the compositions of the present invention are contacted with a subject, for example, human, dog, cat, gerbil, ferret, hamster, horse, pony, donkey, mule, sheep, goat, pig, cow, bull, steer or heifer by spraying a spray or aerosol or application of a liquid, gel, powder, paste, ointment, cream, surface coating, soap, dry residue, lotion or wipe comprising the compositions of the present invention.

In one embodiment of methods of the present invention, the subject is a human.

In one embodiment of methods of the present invention, the subject is a household pet, such as, a dog, cat, bird, rat, mouse, ferret, gerbil or hamster.

In one embodiment of methods of the present invention, the subject is a farm animal, such as, a horse, pony, donkey, mule, lama, alpaca, emu, pig, bird, cow, bull, steer, heifer, sheep, or goat.

In one embodiment, the present invention is a method for the modification of insect and/or arachnid behaviour at a locus causing, for example:
the insect, and/or arachnid to be repelled from the locus prior to, for example, biting/stinging a subject or eating/damaging a plant; and/or
the insect, and/or arachnid to release itself from a subject after biting said subject and prior to completion of it's blood meal and thus reducing the transmission of a pathogen;
   the method comprising contacting said insects and/or arachnids, a locus at which modification is desired, or a subject with an effective amount of a composition of the present invention.

As used herein, the terms “Modification” or “Modify” include causing minor, partial, median and/or major changes in, for example, insect behaviour.

As used herein, the term modification of an insect and/or arachnid “Behaviour” includes modification of their mental/physical actions as well as their biological and/or physiological responses.

In one embodiment, the present invention is a method for the modification of, for example, a biochemical cement which an insect and/or arachnid, for example, a tick uses to anchor themselves to a bite site of a subject, the method comprising contacting said insects and/or arachnids, a locus at which modification is desired, or a subject with an effective amount of a composition of the present invention. In one embodiment of the present invention, the methods of the present invention rapidly dislodge embedded ticks from a subject without the need for mechanical intervention.

In one embodiment of the present invention, the insect and/or arachnid is a flea, midge, mosquito, horse-fly, gnat, louse, tick, or mite.

In one embodiment of the present invention, the insect and/or arachnid is a flea, midge, mosquito, horse-fly tick, or mite.

In one embodiment of the present invention, the insect and/or arachnid is a tick or mite.

In one embodiment of the present invention, the insect and/or arachnid is a mosquito.

In one embodiment of the present invention, the insect and/or arachnid is a tick.

In one embodiment the present invention is a method of treating and/or preventing a disease in a subject in need thereof, wherein said disease is caused by transmission of a pathogen from a hematophagous organism to said subject, the method comprising contacting said hematophagous organism, a locus at which modification is desired or said subject, with a therapeutically effective of an emulsion or a reactive emulsion or composition of the present invention and a pharmaceutically acceptable diluent or carrier.

As used herein, the terms “Treat”, “Treatment” and “Treating” refer to medical (therapeutic), non-medical treatments and prophylactic treatments. For example, therapeutic treatments includes the reduction, suppression or amelioration of the progression, severity and/or duration of a condition, or the amelioration of one or more symptoms (including, one or more discernible symptoms) of a condition, resulting from the administration of one or more compositions of the present invention. In specific embodiments, the therapeutic treatment includes the amelioration of at least one measurable physical parameter of a condition. In other embodiments, the therapeutic treatment includes the inhibition of the progression of a condition, either physically by, e.g., stabilization of a discernible symptom, physiologically by, e.g., stabilization of a physical parameter, or both. Non-medical treatments include, for example, treating an infestation by reducing the number or completely eliminating, for example, an arthropod from a locus. The terms “prophylaxis” or “prophylactic use” and “prophylactic treatment” as used herein, refer to preventing rather than treating or curing a condition or infestation. As used herein, the terms “Prevent”, “Prevention” and “Preventing” refer to the reduction in the risk of acquiring or developing a given condition or infestation, or the reduction or inhibition of the recurrence or said condition or infestation. As used herein, the term “condition” includes disease, illnesses, sickness, infection, and/or biological (including immunological and physiological) response.

In one embodiment of the methods of the present invention, the subject is a human or non-human animal. In another embodiment the subject is a mammal. In one embodiment, the mammal is a human or non-human mammal. In one embodiment, the mammal is a human. In one embodiment of the present invention, the non-human mammal is a household pet, farm animal or zoo animal. In one embodiment, the non-human animal is a dog, cat, mouse, rat, gerbil, ferret, hamster, bird, horse, pony, donkey, mule, lama, alpaca, emu, sheep, goat, pig, cow, bull, steer, heifer, deer, tiger, cheetah, wolf, monkey, lion, bear, fox, gorilla, or kangaroo. In one embodiment of the present invention, the subject is not a parasite, insect, and/or arachnid. In one embodiment, the subject is not a parasite or arthropod.

In one embodiment, the present invention is a method of treating and/or preventing a disease in a subject in need thereof, wherein said disease is caused by a pathogen transmitted to said subject by an insect, arachnid, and/or parasite the method comprising contacting said parasites, insects and/or arachnids, a locus at which treatment and/or prevention is desired, or said subject, with a therapeutically effective amount of a composition of the present invention.

In one embodiment, the invention is a method of treating and/or preventing a disease in a subject in need thereof, wherein said disease is caused by a pathogen transmitted to said subject by an insect, arachnid, and/or parasite comprising contacting said parasites, insects and/or arachnids, a locus at which treatment and/or prevention is desired, or said subject with a therapeutically effective amount topical composition of the present invention and a pharmaceutically acceptable diluent or carrier.

In one embodiment, the invention is a method of treating and/or preventing a subdermal, intradermal or subcutaneous disease in a subject in need thereof, wherein said disease is caused by a pathogen transmitted to said subject by an insect, arachnid, and/or parasite comprising contacting said parasites, insects and/or arachnids, a locus at which treatment and/or prevention is desired, or said subject with a therapeutically effective amount composition of the present invention and a pharmaceutically acceptable diluent or carrier.

In one embodiment the disease is Anaplasmosis, Human Granulocytic Anaplasmosis, Babesiosis, Borrelia mayonii infection, Borrelia miyamotoi infection, Bourbon virus infection, Colorado tick fever, Alkhurma Hemorrhagic Fever, Ehrlichiosis, Heartland virus, Lyme disease, Powassan disease, Rickettsia parkeri rickettsiosis, Rocky Mountain spotted fever (RMSF), STARI (Southern tick-associated rash illness), Tickborne relapsing fever (TBRF), Tularemia, Rickettsiosis, Tick borne encephalitis, Akabane virus, Schmallenberg, Blue-tongue, Equine infectious anaemia, Anthrax, Trypanosomes (trypanosoma/Chagas), Malaria, Dengue, Yellow fever, Zika virus, Chikungunya, Human Lymphatic Filariasis, Black death, Plague, Bubonic plaque, Rickettsia disease, Tungiasis, Chiggers, Scabies, Typhus fever, Parasite infestation, Crimean-Congo Haemorrhagic Fever, Rickettsiosis, or Yersinia pestis.

In one embodiment the parasite, insect and/or arachnid is flea, midge, mosquito, horse-fly, tsetse fly, sandfly, blackfly, bedbug, assassin bug, gnat, louse, sawfly, ant, spider, tick, mite, scorpion, pseudoscorpion, harvestmen, camel spider, whip spider, or vinegarroon. In one embodiment the insect is a flea, midge, mosquito, horse-fly, tsetse fly, sandfly, blackfly, bedbug, assassin bug, gnat, louse, sawfly, or ant. In one embodiment the Arachnid is a spider, tick, mite, scorpion, pseudoscorpions, harvestmen or vinegarroons. In one embodiment, the flea is Tunga penetrans, chigoe, chigo, chigoe flea, chigo flea, jigger, nigua, sand flea, or burrowing flea. In one embodiment the mite is Sarcoptes scabiei var. hominis, Trombiculidae; harvest mite, berry bug, bush-mite, red bus or scrub-itch mite. In one embodiment the parasite is Loa loa.

In one embodiment, the invention is a method of treating and/or preventing a disease in a subject in need thereof, wherein said disease is caused by a pathogen transmitted by an hematophagous organism to said subject, the method comprising contacting said organism, a locus at which treatment and/or prevention is desired, or said subject, with a therapeutically effective amount of a composition of the present invention.

In one embodiment the disease is Anaplasmosis, Human Granulocytic Anaplasmosis, Babesiosis, Borrelia mayonii infection, Borrelia miyamotoi infection, Bourbon virus infection, Colorado tick fever, Alkhurma Haemorrhagic Fever, Ehrlichiosis, Heartland virus, Lyme disease, Powassan disease, Rickettsia parkeri rickettsiosis, Rocky Mountain spotted fever (RMSF), STARI (Southern tick-associated rash illness), Tickborne relapsing fever (TBRF), Tularemia, Rickettsiosis, Tick borne encephalitis, Akabane virus, Schmallenberg, Blue-tongue, Equine infectious anaemia, Anthrax, Trypanosomes (trypanosoma/Chagas), Malaria, Dengue, Yellow fever, Zika virus, Chikungunya, Human Lymphatic Filariasis, Black death, Plague, Bubonic plaque, Rickettsia disease, Tungiasis, Chiggers, Scabies, Typhus fever, Parasite infestation, Crimean-Congo Haemorrhagic Fever, Rickettsiosis, or Yersinia pestis.

In one embodiment the parasite, insect and/or arachnid is flea, midge, mosquito, horse-fly, tsetse fly, sandfly, blackfly, bedbug, assassin bug, gnat, louse, sawfly, ant, spider, tick, mite, scorpion, pseudoscorpion, harvestmen, camel spider, whip spider, or vinegarroon. In one embodiment the insect is a flea, midge, mosquito, horse-fly, tsetse fly, sandfly, blackfly, bedbug, assassin bug, gnat, louse, sawfly, or ant. In one embodiment the Arachnid is a spider, tick, mite, scorpion, pseudoscorpions, harvestmen or vinegarroons. In one embodiment, the flea is Tunga penetrans, chigoe, chigo, chigoe flea, chigo flea, jigger, nigua, sand flea, or burrowing flea. In one embodiment the mite is Sarcoptes scabiei var. hominis, Trombiculidae; harvest mite, berry bug, bush-mite, red bus or scrub-itch mite. In one embodiment the parasite is Loa.

In one embodiment in the methods of the present invention the compositions treat and/or prevent a subdermal, intradermal or subcutaneous disease in a subject.

In one embodiment in the methods of the present invention the compositions are applied topically to a subject and treat and/or prevent a subdermal, intradermal or subcutaneous disease in said subject.

In one embodiment, the present invention is a method of treating and/or preventing a disease in a subject in need thereof, wherein said disease is caused by transmission of a pathogen from a hematophagous organism to said subject, the method comprising contacting said insects and/or arachnids, a locus at which treatment and/or prevention is desired, or said subject, with a therapeutically effective amount of a composition of the present invention prior to an encounter with said insects and/or arachnids.

In one embodiment, the present invention is a method of treating and/or preventing a disease in a subject in need thereof, wherein said disease is caused by transmission of a pathogen from a hematophagous organism to said subject, the method comprising contacting said insects and/or arachnids, a locus at which treatment and/or prevention is desired, or said subject, with a therapeutically effective amount of a composition of the present invention daily, weekly, bi-weekly, monthly, or annually.

In one embodiment of methods of the present invention, the compositions are contacted with an insect and/or arachnid, locus or subject by spraying a spray or aerosol or application of a liquid, gel, powder, paste, ointment, cream, surface coating, soap, dry residue, lotion or wipe comprising the compositions of the present invention.

In one embodiment of methods of the present invention, the compositions of the present invention are contacted with a subject, for example, human, dog, horse, cat, bird, rat, mouse, ferret, gerbil, hamster, pig, cow, bull, steer and heifer, sheep, or goat by spraying a spray or aerosol or application of a liquid, gel, powder, paste, ointment, cream, surface coating, soap, dry residue, lotion or wipe comprising the compositions of the present invention.

In one embodiment, the invention is a method for controlling a parasite, insect and/or arachnid and treating and/or preventing a disease in a subject transmitted to said subject by said parasite, insect and/or arachnid. In one embodiment of methods of the present invention, the subject is a human.

In one embodiment of methods of the present invention, the subject is a household pet, such as, a dog, horse, cat, bird, rat, mouse, ferret, gerbil or hamster.

In one embodiment of methods of the present invention, the subject is a farm animal, such as, a pig, cow, bull, steer and heifer, sheep, or goat.

In one embodiment the parasite, insect and/or arachnid is flea, midge, mosquito, horse-fly, tsetse fly, sandfly, blackfly, bedbug, assassin bug, gnat, louse, sawfly, ant, spider, tick, mite, scorpion, pseudoscorpion, harvestmen, camel spider, whip spider, or vinegarroon. In one embodiment the insect is a flea, midge, mosquito, horse-fly, tsetse fly, sandfly, blackfly, bedbug, assassin bug, gnat, louse, sawfly, or ant. In one embodiment the Arachnid is a spider, tick, mite, scorpion, pseudoscorpions, harvestmen or vinegarroons. In one embodiment, the flea is Tunga penetrans, chigoe, chigo, chigoe flea, chigo flea, jigger, nigua, sand flea, or burrowing flea. In one embodiment the mite is Sarcoptes scabiei var. hominis, Trombiculidae; harvest mite, berry bug, bush-mite, red bus or scrub-itch mite. In one embodiment the parasite is Loa loa.

In one embodiment of the methods of the present invention, the disease is Anaplasmosis, Human Granulocytic Anaplasmosis, Babesiosis, Borrelia mayonii infection, Borrelia miyamotoi infection, Bourbon virus infection, Colorado tick fever, Alkhurma Haemorrhagic Fever, Ehrlichiosis, Heartland virus, Lyme disease, Powassan disease, Rickettsia parkeri rickettsiosis, Rocky Mountain spotted fever (RMSF), STARI (Southern tick-associated rash illness), Tickborne relapsing fever (TBRF), Tularemia, Rickettsiosis, Tick borne encephalitis, Akabane virus, Schmallenberg, Blue-tongue, Equine infectious anaemia, Anthrax, Trypanosomes (trypanosoma/Chagas), Malaria, Dengue, Yellow fever, Zika virus, Chikungunya, Human Lymphatic Filariasis, Black death, Plague, Bubonic plaque, Rickettsia disease, Tungiasis, Chiggers, Scabies, Typhus fever, Parasite infestation, Crimean-Congo Haemorrhagic Fever, Rickettsiosis, or Yersinia pestis.

In one embodiment of the methods of the present invention, the disease is Lyme disease (Borreliosis), Tick Borne Encephalitis, Alkhurma Haemorrhagic Fever, Colorado Tick Fever, Babesiosis, Crimean-Congo Haemorrhagic Fever, Human Granulocytic Anaplasmosis, Rickettsiosis and Tick Borne Relapsing Fever.

In one embodiment of the methods of the present invention, the disease is Zika virus.

The compounds described herein can be formulated into pharmaceutical compositions that further comprise a pharmaceutically acceptable carrier, diluent, adjuvant or vehicle. In one embodiment, the present invention relates to a pharmaceutical composition comprising a composition and/or emulsion of the invention described herein, and a pharmaceutically acceptable carrier, diluent, adjuvant or vehicle. In one embodiment, the present invention is a pharmaceutical composition comprising an effective amount of an emulsion or a reactive emulsion of the present invention or a pharmaceutically acceptable salt thereof and a pharmaceutically acceptable carrier, diluent, adjuvant or vehicle. Pharmaceutically acceptable carriers include, for example, pharmaceutical diluents, excipients or carriers suitably selected with respect to the intended form of administration, and consistent with conventional pharmaceutical practices.

A pharmaceutically acceptable carrier may contain inert ingredients which do not unduly inhibit the biological activity of the compounds. The pharmaceutically acceptable carriers should be biocompatible, e.g., non-toxic, non-inflammatory, non-immunogenic or devoid of other undesired reactions or side-effects upon the administration to a subject. Standard pharmaceutical formulation techniques can be employed.

The pharmaceutically acceptable carrier, adjuvant, or vehicle, as used herein, includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutically acceptable compositions and known techniques for the preparation thereof. Except insofar as any conventional carrier medium is incompatible with the compounds described herein, such as by producing any undesirable biological effect or otherwise interacting in a deleterious manner with any other component(s) of the pharmaceutically acceptable composition, its use is contemplated to be within the scope of this invention.

An “Effective amount” includes a “Therapeutically effective amount” and a “Prophylactically effective amount”. The term “Therapeutically effective amount” refers to an amount effective in treating and/or ameliorating a condition or infestation. The term “Prophylactically effective amount” refers to an amount effective in preventing and/or substantially lessening the condition or infestation. Specific examples of effective amounts are described herein.

### Dosage

In one embodiment of the present invention, the compositions of the present invention comprise between 2% by weight and 60% by weight of an emulsion or a reactive emulsion of the present invention containing no less than 1% by weight and no more than 25% by weight of oil phase wherein the oil phase is no less than 40% by weight of free fatty acid and no more than 60% by weight triglyceride.

In one embodiment of the present invention, the compositions of the present invention are applied in an amount sufficient to cover the insect or the locus which is the intended target of the insect or the area of a subject affected by hematophagous or parasitic insects.

In one embodiment of the present invention, the insect and/or arachnid is a flea, midge, mosquito, horse-fly, gnat, louse, tick, or mite.

In one embodiment of the present invention, the insect and/or arachnid is a flea, midge, mosquito, horse-fly tick, or mite.

In one embodiment of the present invention, the insect and/or arachnid is a tick or mite.

In one embodiment of the present invention, the insect and/or arachnid is a mosquito.

In one embodiment of the present invention, the insect and/or arachnid is a tick.

In one embodiment present invention is a method for suppression, treatment and/or prevention of a physiological and/or immunological response of a subject in need thereof to a bite and/or a sting from an insect and/or arachnid, the method comprising contacting said insects and/or arachnids, a locus at which treatment and/or prevention is desired, or said subject, with a therapeutically effective amount of composition of the present invention and a pharmaceutically acceptable diluent or carrier.

In one embodiment present invention is a method for suppression, treatment and/or prevention of a physiological and/or immunological response of a subject in need thereof to a bite from a hematophagous organism, the method comprising contacting said hematophagous organism, a locus at which treatment and/or prevention is desired, or said subject, with a therapeutically effective amount of a composition of the present invention.

In one embodiment the subject is a human or non-human animal. In another embodiment the subject is a mammal. In one embodiment, the mammal is a human or non-human mammal. In one embodiment, the mammal is a human. In one embodiment of the present invention, the non-human mammal is a household pet, farm animal or zoo animal. In one embodiment, the non-human animal is a dog, cat, mouse, rat, gerbil, ferret, hamster, bird, horse, pony, donkey, mule, lama, alpaca, emu, sheep, goat, pig, cow, bull, steer, heifer, deer, tiger, cheetah, wolf, monkey, lion, bear, fox, gorilla, or kangaroo. In one embodiment of the present invention, the subject is not a parasite, insect, and/or arachnid.

In one embodiment of the methods of the present invention, the compositions of the present invention suppress, treat and/or prevent a physiological and/or immunological response of a subject by modification of the biochemistry of tick saliva and/or other secretion.

In one embodiment of the methods of the present invention, the compositions of the present invention suppress, treat and/or prevent a subject's physiological and/or immunological response to an insect and/or arachnid bite and/or sting, wherein the physiological and/or immunological response is, for example, inflammation; itchiness; erythema; hives; redness; pain; vasoconstriction; platelet aggregation; degranulation of mast cells; release of dendritic cells; activation of fibroblasts, fibrinogen, bradykinins, histamines, or chemoattractant (chemokines or leukotrienes); transport of neutrophils or monocytes; or generating antigen specific antibodies or T lymphocytes.

In another embodiment of the methods of the present invention, the compositions of the present invention suppress, treat and/or prevent a subject's physiological and/or immunological response to a bite or sting from an insect and/or arachnid, such as, a flea, midge, mosquito, horse-fly, tsetse fly, sandfly, blackfly, bedbug, assassin bug, gnat, louse, sawfly, ant, spider, tick, mite, scorpion, pseudoscorpion, harvestmen, camel spider, whip spider, or vinegarroon.

In another embodiment of the methods of the present invention, the compositions of the present invention suppress, treat and/or prevent a subject's physiological and/or immunological response to a bite from an insect and/or arachnid, such as, a flea, midge, mosquito, horse-fly, tsetse fly, sandfly, blackfly, bedbug, gnat, louse, spider, tick, or mite.

In another embodiment of the methods of the present invention, the compositions of the present invention suppress, treat and/or prevent a subject's physiological and/or immunological response to a bite from an insect and/or arachnid, such as, a flea, midge, mosquito, horse-fly, gnat, louse, tick, or mite.

In another embodiment of the methods of the present invention, the compositions of the present invention suppress, treat and/or prevent a subject's physiological and/or immunological response to a bite from an insect, such as, a flea, midge, mosquito, horse-fly, gnat, or louse.

In another embodiment of the methods of the present invention, the compositions of the present invention suppress, treat and/or prevent a subject's physiological and/or immunological response to a bite from a mosquito.

In another embodiment of the methods of the present invention, the compositions of the present invention suppress, treat and/or prevent a subject's physiological and/or immunological response to a bite from an arachnid, such as, a tick or mite.

In another embodiment of the methods of the present invention, the compositions of the present invention suppress, treat and/or prevent a subject's physiological and/or immunological response to a bite from a tick.

In one embodiment of the methods of the present invention, the compositions of the present invention suppress, treat and/or prevent a subject's physiological and/or immunological response to a sting from an insect and/or arachnid, such as, spider, scorpion, or ant.

In one embodiment, the present invention is a method of suppressing, treating and/or preventing a physiological and/or immunological response in a subject in need thereof, wherein said disease is caused by transmission of a pathogen from a hematophagous organism to said subject, the method comprising contacting said insects and/or arachnids, a locus at which treatment and/or prevention is desired, or said subject, with a therapeutically effective amount of a composition of the present invention ad libitum.

In one embodiment, the present invention is a method of suppressing, treating and/or preventing a physiological and/or immunological response in a subject in need thereof, wherein said physiological and/or immunological response is caused by a bite or sting from a hematophagous organism to said subject, the method comprising contacting said insects and/or arachnids, a locus at which treatment and/or prevention is desired, or said subject, with a therapeutically effective amount of a composition of the present invention prior to an encounter with said insects and/or arachnids.

In one embodiment, the present invention is a method of suppressing, treating and/or preventing a physiological and/or immunological response in a subject in need thereof, wherein said physiological and/or immunological response is caused by a bite or sting from a hematophagous organism to said subject, the method comprising contacting said insects and/or arachnids, a locus at which treatment and/or prevention is desired, or said subject, with a therapeutically effective amount of a composition of the present invention once, twice or more.

In one embodiment, the present invention is a method of suppressing, treating and/or preventing a physiological and/or immunological response in a subject in need thereof, wherein said physiological and/or immunological response is caused by a bite or sting from a hematophagous organism to said subject, the method comprising contacting said insects and/or arachnids, a locus at which treatment and/or prevention is desired, or said subject, with a therapeutically effective amount of a composition of the present invention daily, weekly, bi-weekly, monthly, or annually.

In one embodiment of methods of the present invention, the compositions are contacted with an insect and/or arachnid, locus or subject by spraying a spray or aerosol or application of a liquid, gel, powder, paste, ointment, cream, surface coating, soap, dry residue, lotion or wipe comprising the compositions of the present invention.

In one embodiment of the methods of the present invention, the compositions of the present invention:
prevents and/or treats a disease at, for example, a bite site on a subject by, for example:
   modifying the behaviour of an arthropod, such as:
      controlling (repelling and/or killing) an arthropod;
      modifying biological chemistry of an arthropod, for example:
         dislodges embedded arthropods from a locus,
      controlling or killing an insect, arachnid or a pathogen sub dermally, intradermally or subcutaneously; and
   prevents and/or treats a physiological and/or immunological response of, for example, a subject to a bite from an arthropod.

### Methods and Materials

Methods for the manufacture of emulsions of antiparasitic, insecticidal and arachnidicidal compositions

In one embodiment the present invention is a method for the manufacture of the emulsions of the present invention as described herein.

A manufacturing method for a free fatty acid oil in de-lipidised lecithin amphipath is described in WO 2011/061237. Similar methods may be employed to construct emulsions of the present invention. In one embodiment of the methods of the present invention, a secondary high pressure homogenisation step is employed in addition to the methods of WO 2011/061237, to reduce mean particle (emulsion droplet) size to less than 1 micron, preferably in the region of between 0.8 and 0.6 micron, and preferably still less than 0.5 micron.

In one embodiment of the methods of the present invention, an oil phase is constructed by blending one or more free fatty acids with or without a neutral tri-glyceride added and dispersing this in a pre-hydrated suspension of amphipath using a laboratory homogeniser such as an Ultra Turax Model T-25 (IKA Works, NC 28405, USA), fitted with S25N-25G dispersing tool running at 6,000 RPM. Emulsification methods are well known to those skilled in the art.

In one embodiment, the emulsions of the present invention have a mean particle (droplet) size of between 1 and 0.5 microns.

In one embodiment, the emulsions of the present invention have a mean particle (droplet) size of between 1 and 0.5 microns as measured using a Malvern Mastersizer 3000 with Malvern Hydro EV Sampler running on software version 3.7.

In one embodiment, the emulsions of the present invention form the Ultra Turax as described above will have a mean particle (droplet) size of between 1 and 0.5 microns as measured using a Malvern Mastersizer 3000 with Malvern Hydro EV Sampler running on software version 3.7.

In one embodiment, emulsions of the present invention have a mean particle (droplet) size greater than 1 micron. In another embodiment, the emulsions of the present invention have a mean particle (droplet) size of less than 1 micron.

Emulsions with mean droplet size greater than 1 micron will have anti-arthropod, anti-arthropod borne infection, anti-parasitic, insecticidal and arachnidicidal activities. However, emulsions of the present invention, which undergo a secondary homogenisation step to reduce the mean droplet size to less than 1 micron have unexpectedly greatly enhanced efficacy and stability with comparison to the prior art emulsions.

In one embodiment of the methods of the present invention, a secondary homogenisation step is performed using a GEA Niro Soave Panda Plus 200N high pressure homogeniser (or similar) running at 1000 bar pressure and operated according to the manufacturer's instructions. In one embodiment, one or more homogenisation steps (passes through the machine) are used to achieve adequate reduction of droplet size.

In one embodiment, the free fatty acid used in the oil phase is selected from Caproic, Caprylic, Capric, Undecylenic, Lauric, Myristic, Palmitic, Stearic, Oleic, Palmitoleic, Linoleic, linolenic and arachidonic acid, preferably Caproic, Caprylic, Capric undecylenic and Oleic, and more preferably still Caprylic and Capric acid. Suitably pure grades of Caprylic and Capric acid are available from Merck, Germany.

In one embodiment, the free fatty acid oils are diluted with a neutral triglyceride oil. Suitable oils include those of vegetable origin such as olive, peanut, Canola and Coconut. Synthetic triglycerides may also be used and these are preferable for quality reasons. Synthetic triglycerides include those commercially available from Cremer Oleo GmBH, Hamburg Germany marketed under the Miglyol range. Mglyol 812N is a mixed Capric Caprylic Triglyceride and Miglyo 818N is a Caprylic triglyceride. Miglyol 812N is preferred.

In one embodiment of the methods of the present invention, a neutral triglyceride is used to dilute the free fatty acid oil phase. In one embodiment, the triglyceride constitutes between 5% and 60% by weight of total oil (oil phase), preferably the neutral triglyceride should be between 5% and 30% by weight more preferably between 10% and 20% of the total oil (oil phase).

In one embodiment, the total oil (oil phase) is from 2% to 40% of the emulsion of the present invention by weight, preferably 5% to 35% and more preferably 10% to 20%.

In one embodiment of the methods of the present invention, the emulsions further comprise a surfactant. In one embodiment, the surfactant is used to improve dispersion of the oil in the hydrated amphipath. Suitable surfactants may be selected from Anionic, cationic, non-ionic or amphoteric surfactants. Anionic surfactants include salts of free fatty acids such as sodium oleate. Cationic surfactants include lecithin and its constituent phosphatides. Examples of amphoteric surfactants are betaine and sulpho-betaine. Non-ionic surfactants include a series of chemical polymers conventionally known as Tween: Tween 20 is polyoxyethylene sorbitan ester; Tween 40 is polyoxyethylene sorbitan monopalmitate while Tween 80 is polyoxyethylene sorbitan mono-oleate. While any surfactant may be used in the construction of the emulsions of this invention lecithin and one of the Tweens is preferred, preferably lecithin and Tween 80.

In one embodiment, the amphipath is an emulsification agent which is amphipathic having opposing aspects which are hydrophilic and lipophilic. Suitable amphipaths may be selected from Lanolin, Ceramide, cholesterol or lecithin. As well as being a cationic surfactant, lecithin is also an amphipath. Lecithin is the preferred amphipath. Lecithin exists naturally as a combination of five separate phosphatides: phosphatidyl choline, phosphatidyl ethanolamine, phosphatidyl serine, phosphatidyl glycerol and phosphatidic acid. The ratio of phosphatides influences the emulsification properties and may be manipulated to modify the characteristics of the emulsions of this invention. Increased concentration of phosphatidyl choline and phosphatidyl ethanolamine are preferred and more preferable lecithins with at least 60% phosphatidyl choline and no more that 5% phosphatidic acid are preferred.

Amphipaths exist naturally conjugated to lipid or oil. In one embodiment of the methods of the present invention, an amphipath is de-lipidised in a manner which leaves its constituent lipophilic sites free to associate with the oil phase. In one embodiment of the methods of the present invention, de-lipidisation is achieved by repetitive suspension in a solvent such as acetone, precipitation and drying of the precipitate by solvent evaporation. Many suitable lecithins are available commercially as extract of egg or soy. In one embodiment, the amphipath used in the methods of the present invention is soy lecithin is which has been de-lipidised to less than 5% by weight of extraneous lipid or oil.

In another embodiment of the methods of the present invention, a chemically modified lecithin is used to improve electro-static repulsion between emulsified oil droplets thereby improving emulsion stability. Suitable modified lecithin include di-palmitoyl phosphatidyl choline (DPPC) and di-palmitoyl phosphatidyl glycerol (DPPG) both available from Lipoid AG, Steinhausen, Switzerland. In one embodiment, chemically modified lecithin comprises no more than 60% by weight of de-lipidised lecithin, preferably between 10% and 50% and more preferably between 20% and 40% by weight of lecithin.

In one embodiment of the methods of the present invention, the emulsions of the present invention further comprise a thickener such as glycerol, Xanthan gum, Guar gum or gum Acacia. In one embodiment of the methods of the present invention, long term stability of the emulsions is improved by the use of a thickener such as glycerol, Xanthan gum, Guar gum or gum Acacia. In one embodiment of the methods of the present invention, the emulsions further comprise synthetic polymers. In one embodiment, synthetic polymers improve the rheology and stability of the emulsions of the present invention. Suitable synthetic polymers include cellulose derivatives such as carboxy methyl cellulose, and the Carbopols from Noveon Polymers, Cleveland, Ohio, USA.

In one embodiment of the methods of the present invention, the emulsions further comprise acidity regulators and buffers. Further stability and efficacy may be achieved through the use of acidity regulators and buffers to maintain a desired pH during application. Suitable buffers are organic acids and salts thereof including sodium and potassium phosphate, lactate, citrate and benzoate.

A representative example of the emulsions of the present invention is presented in Table 1.

TABLE 1

| # | Ingredient | Weight % (g/100 g) |
|---|---|---|
| 20% Oil Phase | | |
| 1 | Caprylic Acid | 16 |
| 2 | Miglyol 812N | 4 |
| 80% water phase | | 0 |
| 3 | Purified Water | 76.65 |
| 4 | Tween 80 | 0.2 |
| 5 | De-lipidised soy Lecithin | 3 |
| 6 | Xanthan Gum | 0.15 |
| | Total | 100 |

Methods of Emulsification are well known to those skilled in the art
For guidance;
Components 3, 4, 5 and 6 are combined and allowed to hydrate for 60 minutes
Components 1 and 2 are mixed and added slowly to the hydrated water phase using a laboratory homogeniser.
Allow a period of I hour to remove bubbles before high pressure homogenisation

The product of Table 1 is a 20% oil emulsion which is incorporated in finished formulations at dose loadings of between 1 and 20%, preferably between 2% and 10% and more preferably between 4% and 8%.

Optionally a lower total oil phase may be used as exemplified in Table 2 where 10% oil with higher neutral tri-glyceride (Miglyol) is demonstrated using Lipoid DPPG to enhance emulsion stability.

TABLE 2

| # | Ingredient: | Weight % (g/100 g) |
|---|---|---|
| 10% Oil Phase | | |
| 1 | Caprylic Acid | 5 |
| 2 | Miglyol 812N | 5 |
| 90% Water Phase | | |
| 3 | Purified water | 88.29 |
| 4 | Tween 80 | 0.15 |
| 5 | De-lipidised soy lecithin | 1.2 |
| 6 | Lipoid DPPG | 0.36 |
| | Total | 100 |

Methods of Emulsification are well known to those skilled in the art
For guidance;
Components 3, 4, 5 and 6 are combined and allowed to hydrate for 60 minutes
Components 1 and 2 are mixed and added slowly to the hydrated water phase using a laboratory homogeniser.
Allow a period of I hour to remove bubbles before high pressure homogenisation

The product of Table 2 is a 10% oil emulsion which is incorporated in finished formulations I dose loadings of between 1% and 20%, preferably 2% to 10% and more preferably between 4% and 8% by weight.

It should be noted that although the dose loadings are the same the product of Table 2 is delivering half the concentration of the functional de-lipidised lecithin and free fatty acid components in Table 1.

In one embodiment of the methods of the present invention, emulsions can be constructed with total oil (oil phase) ranging from 3% to 30% by weight, preferable 5 to 25% and more preferable 10% to 20%.

In one embodiment of the methods of the present invention, the concentration of the oil phase requires adjustment of the concentration of de-lipidised lecithin or other amphipath to maintain stability of the emulsion. In one embodiment of the present invention, the weight ratio of amphipath to oil (free fatty acid and optionally, for example, triglyceride) is between 0.1 to 1.0 or between 0.5 to 1 or 1 to 1 or 1 to 0.5.

In one embodiment of the methods of the present invention, the weight ratio of neutral oil to free fatty acid in the oil phase may be varied between 0% and 60% triglyceride to 100% to 40% free fatty acid in order to modulate the potency and rate of release of amphipathic emulsification agent.

### EXAMPLES

### Arthropods and Methods of Collection

Cockroaches (Dictyoptera) include the large American cockroach, Periplanetta americans, the smaller Oriental cockroach, Blata orientalis, and the smallest German cockroach Blattella germanica.

The mid-sized Oriental cockroach was collected from a disused grain store prior to de-contamination by a pest control company. Cockroaches are easily cared for in a laboratory setting and are frequently used for education and scientific research purposes. A suitable treatise on the laboratory management of cockroaches is available from Chapman and Hall publishers: W. J. Bell 1981; The laboratory management of cockroach. ISBN 978-0-412-23990-8

Fleas (Siphonaptera), of which there are over 2,500 known species the most common of which are the domestic dog and cat flea, Cteonocephalides canis and C. felis

Cat fleas were collected by grooming cats in a pet rescue centre and transported to a laboratory for testing within 1 hour of collection.

Ticks (Ixodida) of which there are some 700 species of hard bodies tick and 200 soft bodies. The most notable species associated with arthropod borne infection include the sheep tick Ixodes Ricinus, the black legged deer tick Ixodes scapularis and the lone star tick Amblyomma Americanum.

Ixodes Ricinus was collected during summer months from a forest area known to hold a heavy density of red deer and known to be infected with Ixodes. Ticks are easily collected by dragging a white sheet over ground where long grass is growing. Ticks will cling on to the underside of the sheet and can be collected from there using a fine brush to transfer them to a plastic container with lid. Freshly collected ticks were transported to a laboratory for testing within 1 hour of collection.

Mosquito (Culicidae) of which there are some 3,500 known species including Anopheles, Culex and Aedes.

Aedes aegypti were obtained from an established colony and maintained in a laboratory using established methods described in detail by S. W. Masters et. al. Rearing Aedes aegypti Mosquitoes in a Laboratory Setting. Lab Animal Sci Prof. 2020 November'; 55 (6): 42-45.

Midge (Diptera, Ceratopogonidae), there are many different species some bite and some do not. Of the biting midge species Culicoides impunctatus is notorious and responsible for transmission of many animal infections.

Maintenance of a laboratory colony of Culicoides is possible as described by J. Boorman (1974) The Maintenance of Laboratory Colonies of Culicoides. Bulletin of Entomological Research 64(3), 371-377. Doi: 10-1017/S0007485300031254.

In practice it is easier to collect midge from an area such as a lake shore known to be affected by swarming midge. The most suitable time is after dark on a summer evening using an electric bulb behind a sheet of fine muslin which will become covered in midge in a short time. Collected midges were transferred to a plastic container using a pipette bulb on the end of a narrow (2 mm diameter) glass Pasteur pipette.

Collection of head lice: Pediculus humanis captis

Head lice were collected by a school hygiene specialist from school children aged 6 years to 10 years by combing hair using a fine toothed comb.

Harvested lice were transported to the laboratory and tested within 3 hours of collection.

### Example 1: Anti-Arthropod Formulation Effect on Cockroach (Blata orientalis)

### Part 1.

The emulsion concentrate from Table 1 was diluted to 5% by weight in sterile distilled water: 5 grams of emulsion concentrate was added to 95 grams of water and stirred to disperse fully. It should be noted that this dilution contains 0.8% caprylic acid, 0.2% Miglyol 812N (neutral triglyceride), 0.01% Tween 80, 0.15% de-lipidised lecithin, and 0.0075% xanthan gum. The diluted emulsion was loaded into a HDPE spray bottle.

A ‘control’ or blank formulation was sterile distilled water in a similar HDPE spray bottle.

20 cockroaches were selected from a batch of 40 collected as described above.

Individual insects were lifted with a plastic tweezers taking care to use the minimum grip necessary to hold the insect without damaging it. Each insect received a single spray of either the test formulation or control (ten each) and placed in individual plastic sample bottles labelled according to the formulation received.

The insects were evaluated after one hour and scored according to observable movement: 1) immovable, was considered dead or paralysed; 2) moribund, showing some signs of movement; 3) alive, walking and moving freely.

Of the ten insects that received test formulation seven were dead and three were moribund after one hour.

All of the ten control group were alive after one hour.

Both groups were observed over a period of ten hours during which only one of the three deemed moribund in the test group was still show some twitching at the end of the ten hours. All of the control group were still alive at the end of the ten hours.

The test was repeated three times with no recovery of moribund insects after 10 hours and was therefore considered to be 100% effective after 1 hour.

### Part 2

The test was repeated in exactly the same way using the formulation in Table 2. Because Table 2 formulation is 10% oil (half that of Table 1) the formulation was diluted to 10% in sterile distilled water to achieve an equivalent concentration.

In this case the results were similar. After 1 hour in the test groups, 9 insects were dead and 1 remained moribund in two replicates and 7 were dead with 3 moribund in the third group. None of the control insects were affected. After 10 hours all moribund insects in test groups were dead and the test was considered to be 100% effective after 1 hour

### Part 3.

In order to get some measure of the dose response, individual insects were weighed before and after being sprayed with the test formulation (5% dispersion of Table 1 in water). It was estimated that each insect was receiving approximately 0.3 grams of formulation.

Individual insects were then treated using a pipette to place 0.3, 0.2 and 0.1 grams of formulation directly onto their backs. A control using water at each volume was also used.

None of the control insects showed any deleterious effect. Insects treated with 0.1 gram of formulation appeared moribund at 2 hours and immobile overnight. Insects treated with 0.2 grams of formulation were immobile at 1 hour and did not recover overnight. Similarly the 0.3 gram dose was immobilised in less than 1 hour and no recovery overnight was detected.

A minimum dose volume of 0.2 grams was determined but it should be noted that these results are an estimate of dose volume only, not dose concentration. The actual dose delivered in 0.2 grams of formulation was 1.6 mg caprylic acid, 0.4. mg Miglyol, 0.02 mg Tween 80, 0.3 mg de-lipidised lecithin and 0.015 mg xanthan gum.

### Example 2: Evaluation of Mode of Action of Test Formulation

The large American cockroach Periplaneta americana were used to evaluate mode of action because of their size which greatly facilitates dissection and post mortem evaluation.

All cockroaches have typical insect anatomy, in particular their breathing apparatus consists of a series of small openings called spiracles located laterally along both sides of their thorax which led to trachea and tracheole disseminated through their body. Breathing relies on a pumping mechanism activated by abdominal muscles. Additionally cockroaches have a mouth, oesophagus, alimentary canal and anal orifice

The emulsion concentrate from Table 1 was diluted to 5% by weight in sterile distilled water containing 1% ponceau red dye. A control was used consisting of water only with 1% ponceau red dye.

Individual insects were sprayed liberally with either test or control and excess fluid allowed to drain off on blotting paper. Insects treated with test formulation were moribund almost immediately and deemed to be immobile (dead) after one hour. Insects treated with control formulation were unaffected and were euthanized by freezing before dissection.

Dissection was performed by a qualified entomologist in full compliance with ethical guidelines.

Upon dissection the dyed formulation was observed to have penetrated the spiracles and travelled throughout the trachea and tracheole. The dyed formulation was also evident in the oesophagus and lower alimentary tract where it had penetrated through the anal orifice. The dyed formulation was not detected in the internal tissue or the fat bodies (energy stores).

There was no evidence of penetration of the dyed control.

The mode of action is considered to be a physical rather than chemical intervention, primarily suffocation due to gross blockade of the breathing apparatus.

One of the unique characteristics of the emulsions of the present invention is that they exert dramatic wetting of hydrophobic surfaces due to phase inversion by the amphipath. The cuticle of most insects is a waxy surface which is extremely hydrophobic (lipophilic) and which repels water preventing ingress at the spiracles. Provided its lipophilic sites are free to associate, delivery of an amphipath to the insect cuticle and spiracles will result in phase inversion of the surface from lipophilic to hydrophilic permitting immediate ingress of aqueous material to the trachea and also the mouth, oesophagus and anal orifice.

### Example 3: Efficacy Against Head Lice Pediculus humanis Captis

The emulsion concentrate from Table 1 was diluted to 5% by dispersing 5 grams in 95 grams of sterile distilled water. This is similar to the test items used in Example 1 and contains the same concentration and ratio of ingredients.

Head lice were collected as described above. Test insects were confirmed to be alive before testing.

Groups of 20 live head lice were placed on sections of cotton gauze in a petri dish which was then flooded with 5 ml of test formulation or 5 ml of control for 10 minutes. Care was taken to ensure that all sections of the gauze and the insects thereon were fully submerged.

After 10 minutes, gauze sections were carefully lifted and transferred to blotting paper to remove excess test or control formulations.

The lice were checked after 1, 2 and 4 hours and scored according to immobile (dead), moribund (some slight movement) or alive (normal movement of limbs).

One hour after the test exposure 62.5% of insects treated with test formulation were immobile or dead, none of the control insects were affected (all alive). Two hours after the test exposure the mortality in the test group had increased to 66.6 and again all control insects were alive.

Monitoring was discontinued after 4 hours because preliminary tests revealed that ex-Vivo head lice die relatively quickly when removed from their source of a blood meal and insects in the control group will start to die off after 4 to 6 hours either from starvation or desiccation.

TABLE 3
Percent mortality of head lice after 10 minute exposure

| Hours post exposure | 1 | 2 | 4 |
|---|---|---|---|
| Control | 0 | 0 | 0 |
| Test | 62.5 | 66.6 | 64.2 |

In this example a 10 minute exposure of the 5% aqueous dispersion of test formulation achieved greater than 60% mortality of head lice under the test conditions. Greater efficacy may be anticipated In Vivo use or with higher concentrations over longer exposure times and with optional excipients designed to enhance physical contact with the insects.

### Example 4

Mode of Action on Head Lice Pediculus humanis Captis

In a manner similar to that used to evaluate mode of action against cockroach in Example 1, the test formulation was a 5% dispersion of the emulsion concentrate from Table 1 in sterile distilled water containing 1% Ponceau Red dye. The control was ponceau red dye in water only.

Test insects were first confirmed to be alive and then placed on cotton gauze sections and immersed in test or control for one hour after which they were rinsed with clean water, dried on blotting paper and examined under a microscope.

There was no dye visible in or on the control insects.

The dye as not particularly visible in the spiracles or the trachea of the test insects but significant amounts had been taken up in the head capsule and the abdominal haemocoel. The mouth parts of the louse (stylets) are normally retracted and waterproof except when feeding. It was evident that the surfactant properties of the formulation had breached the hydrophobic nature of the mouth and entered the head capsule in significant amounts. Similarly the cloaca which serves as a common orifice for anus and reproductive organs is normally closed and resistant to ingress. The abdominal haemocoel was observed to be full of dyed formulation which can only have entered through the cloaca.

The mode of action was deemed to be due to physical damage caused by ingress of the formulation through natural orifices.

### Example 5: Efficacy Against Tick Ixodes ricinus

In this Example a prototype gel was used incorporating the emulsion from Table 1 with added glycerol and Xanthan gum to achieve a slightly sticky formulation that will stay in place when applied to insects that may be residing or embedded in skin, hair or fur. Sodium citrate was also used here as a buffer. The combination of excipients was shown to improve efficacy by controlling pH and increasing contact with the insect's surface.

Sterile distilled water, a 5% aqueous dispersion of the emulsion and the combination of excipients without the emulsion have been used as controls.

TABLE 5
Gel formulation for use against ticks

| | | Weight % |
|---|---|---|
| | | (grams/ |
| # | Ingredient | 100 grams) |
| 1 | 50 mM sodium citrate | 84 |
| | buffer pH 5.0 | |
| 2 | Glycerol | 10 |
| 3 | Xanthan Gum | 1 |
| 5 | Emulsion from Table 1 | 5 |
| | Total | 100 |

Notes
The emulsion concentrate is delivering the same amount of ingredients as listed in Example 1:
0.8% caprylic acid, 0.2% Miglyol 812N (neutral triglyeride), 0.01% Tween 80, 0.15% de-lipidised lecithin, and 0.0075% xanthan gum.
Sodium citrate is prepared using 50 mM sodium citrate to adjust the pH of 50 mM citric acid to 5.0 using a pH meter
Glycerol is added to sodium citrate buffer and stirred to dissolve fully
Xanthan gum is added to the glycerol/sodium citrate solution with the aid of a laboratory blender taking care not to trap air bubbles
When the xanthan gum is fully hydrated the emulsion concentrate from Table 1 is added and stirred to mix thoroughly
Excipient Blank:
Prepared as above with water adjusted from 84% to 89% and no Emulsion added
Active Control
Based on a 5% dispersion of the emulsion from Table 1 in sterile distilled water (5 grams in 95 grams).
Water control:
Sterile distilled water only

Although the formulation is sufficiently sticky to adhere to skin, hair and fur it is pourable and very easy to rinse off with water.

Ticks were collected from the field in the manner described above and used in test within one hour of collection.

Ticks were assembled in small (35 mm) petri dishes in lots of 5 individuals and all confirmed to be alive before exposure to test items.

Plates containing insects were then flooded with either test gel, excipient blank, active control, or water control.

After 5 minutes exposure plates were drained by stretching cotton gauze over the plate to retain insects before inverting it to drain. A volume of sterile distilled water was added to the plates to dilute residual test item, re-inverted to drain and covered with blotting paper to absorb excess liquid.

Insects were evaluated at 1, 2, 4 hours post exposure and overnight (18 hours) and scored according to movement: immobile=dead; moribund=some movement of limbs; alive=obvious movement

TABLE 6
Efficacy of 5% emulsion in prototype Gel (active),
blank gel, active control and water control.
5 minute exposure, wash, drain and evaluate

| Hours post Exposure | 1 | 2 | 4 | O/night |
|---|---|---|---|---|
| Active Gel | | | | |
| Dead | 4 | 5 | 5 | 5 |
| Moribund | 1 | 0 | 0 | 0 |
| Alive | 0 | 0 | 0 | 0 |
| Gel blank | | | | |
| Dead | 0 | 0 | 1 | 1 |
| Moribund | 1 | 1 | 0 | 0 |
| Alive | 4 | 4 | 4 | 4 |
| Active Control | | | | |
| Dead | 2 | 2 | 4 | 5 |
| Moribund | 2 | 2 | 1 | 0 |
| Alive | 1 | 1 | 0 | 0 |
| Water Control: | | | | |
| Dead | 0 | 0 | 0 | 0 |
| Moribund | 0 | 0 | 0 | 0 |
| Alive | 5 | 5 | 5 | 5 |

One hour after a 10 minute exposure one of the five insects showed some signs of movement and four were dead. After two hours all 5 test insects exposed to active gel were dead.

The gel blank had a deleterious effect on one of the five test insects over two hours but four were unaffected. Two hours later and overnight, the affected insect was dead but all of the other four started moving when poked with a tweezers.

Two of the five insects exposed to active control (emulsion in water no gel) were dead within one hour, two were moribund (some movement of limbs) and one appeared to be unaffected.

After four hours, the two moribund insects had died (four dead in total) and one remained moribund but also died overnight.

The water control had no effect on any of the five exposed insects. When left for long periods ticks stop moving of their own accord but unless dead or moribund they will start moving again when touched with a solid object such as the end of tweezers.

A further test was carried out using the active gel and gel blank, without washing—the test items were left in contact with the insects and observations were made over a 2 hour period, the results are presented in Table 7

TABLE 7
Efficacy of 5% emulsion in prototype gel (Active gel) and gel blank
No wash off

| Minutes post exposure | 10 | 30 | 60 | 120 |
|---|---|---|---|---|
| Active Gel - no wash off | | | | |
| Dead | 5 | 5 | 5 | 5 |
| Moribund | 0 | 0 | 0 | 0 |
| Alive | 0 | 0 | 0 | 0 |
| Gel Blank - no wash off | | | | |
| Dead | 0 | 0 | 0 | 0 |
| Moribund | 0 | 0 | 1 | 1 |
| Alive | 5 | 5 | 4 | 4 |

Ten minutes after exposure all five insects exposed to the active gel were dead

Sixty minutes after exposure to the gel blank one of the five insects was moribund and remained so after 120 minutes, the other four were unaffected.

In summary: A 5% aqueous dispersion of the emulsion in Table 1 has a potent effect on ticks (Ixodes Ricinus). Potency is amplified slightly using gel excipients to improve adherence to the insect and an organic acid buffer to maintain acidity. The buffered gel has very little effect without the emulsion.

### Example 6: In Vivo Assessment Against Tick (Ixodes Ricinus)

The protective effect of the gel formulation in Example 5 was demonstrated and also the utility of the same formulation in dislodging embedded tick from a human subject.

The gel formulation from Example 5, table 5 was used in this Example.

A one inch wide ring of the gel was applied to the upper leg (thigh) of a human subject, the “treated area”, leaving a three inch diameter inner circle with no gel.

One freshly collected tick was carefully placed in the middle of the inner circle using a plastic tweezers and allowed to meander at will. Ticks move relatively quickly and the test insect made its way onto the treated area within a minute of placement. As soon as the insect entered the treated area it started showing signs of distress-short jerking movements with front legs waving erratically. Location movement stopped after about 2 minutes and the tick turned itself upside down after 4 minutes. All movement ceased after 8 minutes and the insect was removed to a petri-dish where it was observed for a further four hours and showed no signs of recovery during that time.

The test was repeated three times with similar results.

Three freshly collected ticks were then placed on clean untreated human subject's skin and retained in place by taping an inverted 35 mm petri-dish over the insects. The insects wandered inside the isolated area for about 10 minutes before one bit and quickly became embedded in the subject's skin. The petri-dish was removed along with the other two ticks and the embedded tick was left in place for two hours to become established. After the two hours lapsed time an aliquot of about 0.5 ml of the 5% emulsion gel (Example 5, Table 5), was applied to the tick, and spread about 1 cm diameter around the bite site and left for 10 minutes. After 10 minutes, gentle rotation of a finger caused the tick to come away from the bite site. The tick was placed in a plastic container and observed for a further four hours during which time no movement was observed. Unlike ticks removed with a tweezers, the tick was anatomically intact: the scutum and mouth parts were still attached.

It is known that ticks secrete a “cement” like substance to weld themselves into a bite site while feeding. It is also known that ticks secrete substances to dissolve the cement when they need to release on completion of feeding. It is thought that application of the emulsion gel on a feeding tick cause it to dissolve its own fixing cement in an (albeit futile) attempt to escape—hence the ease of removal. A more immediate reduction in the tenacity of the fixing cement may be solubilisation by the surfactant effect of the emulsion.

The test was repeated three time with similar results.

The above tests were carried out during summer months in a forest area noted for being infested with tick Ixodes Ricinus. It is not unusual for forestry workers in the area to have six or more ticks embedded in their legs at the end of a working day. Conventional removal of embedded tick is with the use of tweezers. An observation that has been made is the absence of ongoing irritation with the gel removal in comparison to the hot itchy bite sites commonly experienced when ticks are removed with tweezers.

The observed reduction of itch and inflammation in a tick bite site treated with emulsion is thought to be due to Mast Cell blockade by the amphipathic aspect released subdermally. As demonstrated in WO 2011 061237, de-lipidised lecithin will coat surfaces of mammalian cells and block adhesion of potential pathogens by phase inversion—the normally lipophilic cell surface becomes hydrophilic. While not wishing to be bound by the explanation it is thought that a similar mechanism of action blocks the surface of mast cell located in the vicinity of the bite site, preventing de-granulation in response to insect saliva and consequential itch and inflammatory response.

### Example 7: In Vivo Efficacy Against Midge (Culicoides)

Culicoides is a genus of biting midge in which there are over 1,000 species and much debate about taxonomic validity: impunctatus, sonorensis and scoticus are examples commonly referenced. Regardless of classification all are potential vectors of viral disease in animals and all are notorious for the irritation they cause to humans especially outdoors during late evenings.

As described above Culicoides sp can be collected by trapping them on a muslin screen over an electric light bulb just before dark on summer evenings. Because of the small size it is difficult to determine if they are alive or dead and consequently In Vitro evaluation of efficacy was abandoned.

In Vivo evaluation was carried out using human subjects in an area noted for intense swarming of highland midge (Culicoides impunctatus). In this area midge bites will leave any exposed skin peppered with small red marks which develop within 10 minutes of the bite.

The gel described in Example 5 Table 5 containing 5% emulsion from Table 1 was used in this evaluation.

### Human Testing

Two human subjects dressed in appropriate head and face coverings applied the gel to one forearm and allowed it to dry for 15 minutes, following which they exposed both forearms to a midge infested area for 15 minutes. At the end of the exposure period both arms were covered and the subjects returned to a less hostile environment. 15 minutes after the exposure period subject's arms were evaluated for midge bite marks. Forearms that had been treated with test formulation were almost entirely free of midge bite marks. Untreated forearms, particularly the inner forearm were covered in red spots approximately 2 mm in diameter with a density of approximately one every square inch. Some individuals are more sensitive to midge bite than others. Individuals who spend a lot of time outdoors can become tolerant to midge bite and red marks from bites disappear within an hour or so. For individual who are sensitive to midge bite an erythema of up to 10 mm in diameter may develop which is painful and may persist for several days.

Two subjects who were particularly sensitive to midge bite exposed their forearms for 3 minutes to acquire a number of bites on each arm. Following confirmation of bites (erythema developing), a thin film of test gel was applied to some of the bites on each arm and other were left untreated. Surprisingly bite marks that were treated with gel subsided in a matter of hours while those untreated went on to develop a characteristic red weal which lasted for several days.

### Animal Testing

Horses suffer greatly from midge bite during summer months and as with humans some individuals are more afflicted than others. Sensitive horses may develop Allergic Insect Bite Hypersensitivity (sweet itch) which, if left unattended, may be so severe as to cause the animal to break its own skin by biting, scratching or rubbing itself against any solid object available to it. Affected areas are usually behind the ears, groin, tail dock and croup of the hindquarters. Early stage sweet itch is evident as areas of hair loss with inflamed skin which may become crusty and weeping, and susceptible to secondary bacterial infection. The progress of sweet itch is exacerbated in patches of hair loss which serve as focal points for amplified midge biting due to more accessible skin.

Three horses susceptible to seasonal Sweet Itch were monitored during a summer season to detect early stage Sweet Itch-inflamed irritated hairless patches. As soon as these patches were observed they were treated twice daily by sponge application of the gel formulation from Table 5 in Example 5, selected patches were left untreated for comparison.

After two days treatment it was evident from the horses behaviour that the treated areas had become less irritated, untreated areas were still subject to biting and scratching. At the end of ten days skin areas treated with active gel had healed and showed signs of hair recovery. There was no detectable improvement in untreated areas which had in fact become larger and more inflamed over the course of ten days. Treatment of previously untreated areas on affected hoses commenced and continued through the summer season. The severity of Sweet Itch was significantly reduced during the summer season, none of the affected horses progressed to broken skin and none showed any sign of secondary infection.

It was evident that application of the prototype gel had a dual effect which paralleled those observed in human subjects: first, midge were repelled from treated areas and stopped biting there; second, the inflammatory response to the midge bite (allergic response to midge saliva) was suppressed and the urge to scratch (itch) was ameliorated resulting in healing and recovery of hair growth.

As with tick bite in Example 6 the observed reduction of itch and inflammation is thought to be due to Mast Cell blockade by the amphipathic aspect of the emulsion released subdermally. As demonstrated in WO 2011 061237, de-lipidised lecithin will coat surfaces of mammalian cells and block adhesion of potential pathogens by phase inversion—the normally lipophilic cell surface becomes hydrophilic. While not wishing to be bound by the explanation it is thought that a similar mechanism of action blocks the surface of mast cells located in the vicinity of the bite site, preventing de-granulation in response to allergens in the insect saliva with consequential release of histamine, itch and inflammatory response.

### Example 8: Efficacy Against Fleas (Cteonocephalides felis and C. canis)

Cat fleas were collected from a pet rescue centre as described above.

A water dispersion of 5% emulsion from Table 1 was constructed by adding 5 grams of emulsion concentrate to 95 grams of sterile distilled water and mixing thoroughly. The emulsion diluent was placed in a hand held HDPE spray bottle for application.

A similar spray bottle containing sterile distilled water only was used as a control.

Groups of twenty fleas were placed in a plastic container and all confirmed to be alive immediately before treatment. Groups of 20 were sprayed with either the 5% test item or water control and observed for movement using the scoring system previously described: immobile=dead; some movement of limbs=moribund; full movement=alive.

The effect of the 5% test emulsion was almost immediate with all test insects scored as dead or moribund. The water control had no effect on any of the test insects over a 24 hour period.

The results are presented in Table 8 below.

TABLE 8
Efficacy of 5% Emulsion from Table 1 against cat flea

| Exposure Time | Immobile | Moribund | Alive | Mortality |
|---|---|---|---|---|
| 5% Emulsion | | | | |

| | | | | | |
|---|---|---|---|---|---|
| 1 | min | 14 | 16 | 0 | 100% |
| 5 | min | 20 | 0 | 0 | 100% |
| 15 | min | 20 | 0 | 0 | 100% |
| 30 | min | 20 | 0 | 0 | 100% |
| 60 | min | 20 | 0 | 0 | 100% |
| 24 | Hours | 20 | 0 | 0 | 100% |

Water Control

| | | | | | |
|---|---|---|---|---|---|
| 1 | min | 0 | 0 | 20 | 0% |
| 5 | min | 0 | 0 | 20 | 0% |
| 15 | min | 0 | 0 | 20 | 0% |
| 30 | min | 0 | 0 | 20 | 0% |
| 60 | min | 0 | 0 | 20 | 0% |
| 24 | Hours | 0 | 0 | 20 | 0% |

### Example 9: Evaluation of the Individual Components of the Emulsion

In previous Examples the emulsion of Table 1 and Table 2 have been shown to exert very significant insect repellent and insecticidal effect together with subdermal modulation of physiological response to insect saliva. The composition of both Table 1 and 2 are similar with the exception that the ratio of oil is different and Xanthan gum is used in Table 1 and Lipoid di-Palmitoyl-phosphatidylglycerol (DPPG) is used in Table 2

In use as aqueous dispersions (Examples 1, 2, 3, 4 and 8) the emulsions were diluted to achieve similar concentration of total oil (oil phase), but again, the oil ratio differed. In examples 5, 6 and 7 the emulsions was delivered in the form of a gel containing 10% glycerol and 1% Xanthan Gum in 50 mM sodium citrate buffer. In Example 5 the gel blank was shown to have no effect on the test insect, and so it is reasonable to suggest that none of the gel constituents contributed to efficacy.

In order to evaluate the effect of individual components, the higher concentration of each was used, these are marked with an asterisk as presented in Table 9

TABLE 9
Evaluation of the Effect of Individual Components of the Emulsions
Aqueous Concentration in Weight Percent (grams per
100 grams) of individual components

| | | Table 1 diluted | Table 2 diluted |
|---|---|---|---|
| # | Component | 5% W/W | 10% W/W |
| 1 | Caprylic acid | 0.8* | 0.5 |
| 2 | Miglyol 812N | 0.2 | 0.5* |
| 3 | Tween 80 | 0.01 | 0.015* |
| 4 | De-lipidised lecithin | 0.15* | 0.036 |
| 5 | Xanthan gum | 0.0075* | 0 |
| 6 | DPPG | 0 | 0.12* |

Apart from Xanthan Gum and Tween 80 none of the components are water soluble. An aqueous dispersion of de-lipidised lecithin and DPPG is prepared by blending the component with water at the desired concentration and allowing at least one hour to fully hydrate. The dispersion should be shaken before use as there is a tendency to settle out. Neither Caprylic acid or Miglyol are water soluble, dispersions are achieved with the aid of a laboratory homogeniser (Ultra Turax as described in the methods), however these dispersions will co-acervate and settle out within minutes of being formed and so they were used in this example immediately after they were prepared.

The cockroach Blata orientalis was used as described in Example 1 part 3 because of its size, ease of application and predictable response to the formulation.

Pairs of insects were treated by applying 0.3 grams (ml) of test items from Table 9 above directly onto their backs following which individual insects were placed in separate plastic containers and observed over a period of one hour and overnight. A 5% aqueous dispersion of the emulsion from Table 1 and sterile distilled water were used as controls.

After one hour both insects treated with the emulsion control were dead, none of the insects treated with test items or water control appeared to be affected in any way (all alive). When left overnight (18 hours), one of the two insects treated with caprylic acid was dead, the other appeared to be unaffected.

TABLE 10
Evaluation of Insecticidal Effect of individual
components at concentration in use

| | | | 1 hour post | | 18 hours post | |
|---|---|---|---|---|---|---|
| | | | treatment | | treatment | |
| # | Component | Weight % | Alive | Dead | Alive | Dead |
| 1 | Caprylic acid | 0.8 | 2 | | 1 | 1 |
| 2 | Miglyol 812 | 0.2 | 2 | | 2 | |
| 3 | Tween 80 | 0.015 | 2 | | 2 | |
| 4 | De-lipidised lecithin | 0.15 | 2 | | 2 | |
| 5 | DPPG | 0.12 | 2 | | 2 | |
| 6 | Xanthan Gum | 0.0075 | 2 | | 2 | |
| 7 | Emulsion Table 1 | 5.0 | | 2 | | 2 |
| 8 | Water | 100 | 2 | | 2 | |

Apart from Caprylic acid none of the individual components had any effect 18 hours after exposure to the test items. When assembled in an emulsion or a reactive emulsion as per Example 1 and item 7 in Table 10 above, all of the exposed insects were dead in less than one hour.

The efficacy of the combined components when assembled in the form of an emulsion or a reactive emulsion is thought to be due to the use of free fatty acids as depletable oils to provisionally block the lipophilic sites of the amphipath and subsequently free them when the oils is converted to a water soluble soap by pH shift as happens on contact with the surface of the insect.

### Example 10: Efficacy Against Plant Pathogens

Aphids/Greenfly (Hemiptera) and Whitefly (Trialeurodes) were used here to demonstrate the utility of the emulsion against plant pathogens. Collection and laboratory manipulation of White and green fly is not practical and is easily obviated by the fact they commonly infest glasshouses used for cultivation of ornamental plants, roses, vegetables and soft fruit where treatment with test formulation is convenient.

Both Aphids and White Fly are sap-sucking parasites on plants causing significant leaf damage and in addition both are common vectors of plant virus disease. Infestation of glasshouse plants is easily detected by visual inspection of the underside of leaves and the crook between leaf and stem for aphid. Unlike Aphids, Whitefly do fly and tend to disperse in clouds when a plant is disturbed. A particularly heavily infested domestic glasshouse (both whitefly and Aphid) growing tomatoes and strawberries) was selected for testing the formulation.

As described previously, a water dispersion of 5% emulsion from Table 1 was constructed by adding 5 grams of emulsion concentrate to 95 grams of sterile distilled water and mixing thoroughly. The emulsion diluent was placed in a hand held HDPE spray bottle for application. A similar spray bottle containing sterile distilled water only was used as a control.

Individual plants were treated with either 5% emulsion or water control and inspected at hourly intervals. Plants treated with the emulsion were completely clear of infestation within one hour whereas the water control had no effect. The insects were visibly affected by the formulation immediately after treatment and appeared discoloured and contorted.

Two approximately equal sized tomato plants in pots, one treated with emulsion and the other with water control, were placed side by side. The treated plant remained free of Aphid for four days after which some re-infestation from the neighbouring control treated plant was observed. The effect of a single spray treatment under the test conditions gave immediate reduction of infestation and protection from re-infestation for a period of up to four days

Ants (Formicidae) will devastate soft fruit such as strawberries and frequently establish a commensal relationship with Aphids. Ants are known to ‘farm’ aphids by moving them into colonies in order to feed off the honeydew secretions typically seen where infestation is heavy.

Ants will establish nests in soft dry soil in glasshouses. Frequently the entrance hole is adjacent to a concrete pathway to give physical support for the subterranean structure. Digging out individual ants nests is pointless because they will spread and establish new nests.

A dispersion of the emulsion of Table 1 was used at 10% concentration by adding 10 grams of emulsion concentrate to 90 grams of sterile distilled was and dispensing this from a spray bottle similar to those used to treat aphid infestation.

The location of ants nests was determined by visual observation of where individuals were travelling to. Once located the entrance hole and the surrounding ground was saturated with spray of 10% emulsion. Individual ants in the locality were also sprayed. The procedure was repeated twice daily for three days after which no ants were observed entering or leaving the nest entrance. It is thought that ants transiting the sprayed area were affected by the emulsion and died in the nest.

### Example 11: Non-Reactive Emulsion

The emulsion from Table 1 (reactive emulsion) was adjusted by removing the free fatty acid (caprylic) and replacing it on a weight by weight basis with neutral triglyceride (Miglyol 812N), Table 11, to create a non-reactive emulsion. After high pressure homogenisation the concentrate was diluted to 5% by weight with water. The emulsion of Table 1 diluted to 5% in water was used as a positive control and water was used as a blank.

TABLE 11
Composition of Non-Reactive Emulsion

| # | Ingredient | Weight % (g/100 g) |
|---|---|---|
| 20% Oil Phase | | |
| 1 | Caprylic Acid | 0 |
| 2 | Miglyol 812N | 20 |
| 80% water phase | | 0 |
| 3 | Purified Water | 76.65 |
| 4 | Tween 80 | 0.2 |
| 5 | De-lipidised soy Lecithin | 3 |
| 6 | Xanthan Gum | 0.15 |
| | Total | 100 |

Methods of Emulsification are well known to those skilled in the art
For guidance;
Components 3, 4, 5 and 6 are combined and allowed to hydrate for 60 minutes.
Components 1 and 2 are mixed and added slowly to the hydrated water phase using a laboratory homogeniser.
Allow a period of I hour to remove bubbles before high pressure homogenisation

The Oriental Cockroach Blata Orientalis was used to evaluate the efficacy of the non-reactive emulsion and the experimental design was the same as in Example 1. Individual insects were lifted with tweezers and sprayed with either non-reactive test formulation, positive control or water blank, ten insects in each case, and evaluated after 1 hour and 10 hours.

TABLE 12
Comparative Efficacy of Non-Reactive Emulsion
against Cockroach Blata Orientalis

| | | | | |
|---|---|---|---|---|
| Sample/ | | | | |
| Exposure time | Immobile | Moribund | Alive | Mortality |

Non-Reactive Emulsion

| | | | | | |
|---|---|---|---|---|---|
| 1 | hour | 0 | 2 | 8 | 0% |
| 10 | hours | 1 | 0 | 9 | 10% |

Positive Control

| | | | | | |
|---|---|---|---|---|---|
| 1 | hour | 10 | 0 | 0 | 100% |
| 10 | hours | 10 | 0 | 0 | 100% |

Water Blank

| | | | | | |
|---|---|---|---|---|---|
| 1 | hour | 0 | 0 | 10 | 0% |
| 10 | hours | 0 | 0 | 10 | 0% |

In this test the non-reactive emulsion had no effect after 1 hour exposure and an apparent 10% after 10 hours exposure. The positive control was 100% effective after 1 hour and the water blank had no effect.

### Example 12: Reactive Emulsion Used as a Delivery System for Conventional Oil Soluble Insecticides

Many conventional insecticides are oil soluble and many are toxic to humans and animals with additional environmental impact particularly on pollinators and fish As demonstrated here it is possible to incorporate oil soluble insecticides in the oil phase of a reactive emulsion thereby achieving more targeted delivery and amplified potency facilitating lower effective dosing.

As an example Permethrin is a pyrethroid derivative, it is routinely used as a treatment for head lice and scabies in humans for which it is approved by FDA as a 1% solution. Permethrin is effective against adult lice but not their eggs. Although considered safe for skin contact it can have serious adverse effect particularly if accidentally swallowed or because of its low vapour pressure it can be inadvertently inhaled.

Trapping Permethrin in oil droplets in a Reactive Emulsion permits safer and much lower effective dosing.

In Table 13 below, the free fatty acid was reduced to 25% of the oil phase, (“reduced fatty acid” emulsion) but experimental evidence suggest it is enough to allow the emulsion to react and break to free the residual oil phase. Permethrin was added to the oil phase at 2 grams in 20 of total oil (oil phase) which is 2% of the concentrated reactive emulsion. For use in the test the concentrate was diluted to 5% in water, that is 1% oil phase containing 0.1% Permethrin (10% of recommended dose).

TABLE 13

| # | Ingredient | Weight % (g/100 g) |
|---|---|---|
| 20% Oil Phase | | |
| 1 | Caprylic Acid | 5 |
| 2 | Miglyol 812N | 13 |
| 3 | Permethrin* | 2 |
| 80% water phase | | 0 |
| 4 | Purified Water | 76.65 |
| 5 | Tween 80 | 0.2 |
| 6 | De-lipidised soy Lecithin | 3 |
| 7 | Xanthan Gum | 0.15 |
| | Total | 100 |

*Permethrin (CAS# 52645-53-1) was obtained from BOC Agrichemicals, Great Portland Street, London, UK
Methods of Emulsification are well known to those skilled in the art
For guidance;
Components 4, 5, 6 and 7 are combined and allowed to hydrate for 60 minutes.
Components 1, 2 and 3 are mixed (Permethrin needs time to dissolve in the oil) and added slowly to the hydrated water phase using a laboratory homogeniser.
Allow a period of I hour to remove bubbles before high pressure homogenisation

Although basically oil soluble, Permethrin is sparingly soluble in water and a water control was prepared by dissolving 0.1% Permethrin in water at 25 °C.

A Reactive Emulsion control was prepared using the formulation in Table 13 by replacing Permethrin with Miglyol at 15% and free fatty acid at 5%.

Water was used as a blank.

The Oriental Cockroach Blata Orientalis was used to evaluate the efficacy of the non-reactive emulsion and the experimental design was the same as in Example 1. Individual insects were lifted with tweezers and sprayed with either non-reactive test formulation, positive control or water blank, ten insects in each case, and evaluated after 1 hour and 10 hours.

TABLE 14
Efficacy if Permethrin delivered in reduced fatty acid
Reactive Emulsion against Cockroach Blata Orientalis

| | | | | |
|---|---|---|---|---|
| Sample/ | | | | |
| Exposure time | Immobile | Moribund | Alive | Mortality |

Reduced fatty acid Reactive Emulsion with 0.1% Permethrin

| | | | | | |
|---|---|---|---|---|---|
| 1 | hour | 0 | 0 | 10 | 0% |
| 10 | hours | 4 | 1 | 5 | 50% |

0.1% Permethrin in water

| | | | | | |
|---|---|---|---|---|---|
| 1 | hour | | | 10 | 0% |
| 10 | hours | 0 | 3 | 7 | 30% |

reduced fatty acid Reactive Emulsion Control (no Permethrin)

| | | | | | |
|---|---|---|---|---|---|
| 1 | hour | 0 | 0 | 10 | 0% |
| 10 | hours | 0 | 2 | 8 | 20% |

Water Blank

| | | | | | |
|---|---|---|---|---|---|
| 1 | hour | 0 | 0 | 10 | 0% |
| 10 | hours | 0 | 0 | 10 | 0% |

When delivered in reduced fatty acid Reactive Emulsion Permethrin at 0.1% (10% below recommended dose) has 50% efficacy against Blata Orientalis 10 hours after exposure. A 0.1% aqueous solution of Permethrin achieves 30% efficacy. The reduced fatty acid Reactive Emulsion is 20% effective. A water blank is ineffective. The results suggest that delivery of Permethrin in reduced fatty acid Reactive Emulsion amplifies efficacy by 20% over efficacy in water. It is also evident that the efficacy when delivered in reduced fatty acid Reactive Emulsion is amplified by the efficacy of the reduced fatty acid Emulsion on its own.

It is not possible to conduct these tests using higher concentration fatty acid Reactive Emulsion as a carrier because the action rate of the Reactive Emulsion is so much greater than Permethrin. It can be extrapolated that delivery in higher concentration fatty acid emulsion will have significant improved efficacy in practice.

### Example 13: Amplification of Reactive Emulsion Insecticidal Effect Using Amino Acid Derivatives

Two amino acid derivatives were shown to amplify the efficacy of the Reactive Emulsion, these are N-Acetyl Cysteine (NAC) and Pyrrolidone Carboxylic Acid (PCA). Both of these are water soluble and may be added to the water phase of either the emulsion concentrate or the diluted suspension.

N-Acetyl Cysteine, CAS #616-91-1 was obtained in powder form from BOC Sciences, Great Portland Street London, Catalogue number BO689-469 594.

The sodium salt of 2-Pyrrolidone-5-Carboxylic Acid, CAS #54571-67-4 was obtained as a 50% solution in water from Thermo Fisher Scientific, Waltham, Ma, USA catalogue number 232921000.

Both NAC and PCA have slightly basic pH in solution. In one embodiment of the present invention, Reactive Emulsions of this invention are acidic and are kept at or below pH 5.5 to retain reactivity. For this reason 0.1% W/W solutions of NAC and PCA were prepared in 50 mM sodium citrate buffer at pH 5.0 and reduced fatty acid Reactive Emulsion concentrate (Table 15) was added to these at 5% by weight.

NAC and PCA controls were prepared by dissolving 0.1% of each in 50 mM sodium citrate buffer
reduced fatty acid Reactive Emulsions were used to illustrate the additive effect of NAC and PCA because the rate of activity of higher concentration fatty acid Emulsions would swamp the effect.

A 5% by weight suspension of Emulsion concentrate was prepared in 50 mM sodium citrate buffer without either NAC or PCA was prepared as a negative control and 50 mM buffer on its own was used as a blank.

TABLE 15
Reduced Fatty Acid Reactive Emulsion Concentrate
used to evaluate supplementation with NAC or PCA

| # | Ingredient | Weight % (g/100 g) |
|---|---|---|
| 20% Oil Phase | | |
| 1 | Caprylic Acid | 5 |
| 2 | Miglyol 812N | 15 |
| 80% water phase | | 0 |
| 4 | Purified Water | 76.65 |
| 5 | Tween 80 | 0.2 |
| 6 | De-lipidised soy Lecithin | 3 |
| 7 | Xanthan Gum | 0.15 |
| | Total | 100 |

Methods of Emulsification are well known to those skilled in the art
For guidance;
Components 4, 5, 6 and 7 are combined and allowed to hydrate for 60 minutes.
Components 1 and 2 are mixed and added slowly to the hydrated water phase using a laboratory homogeniser.
Allow a period of I hour to remove bubbles before high pressure homogenisation

The Oriental Cockroach Blata Orientalis was used to evaluate the efficacy of the NAC and PCA supplemented reactive emulsion and the experimental design was the same as in Example 1. Individual insects were lifted with tweezers and sprayed with either NAC or PCA supplemented test formulations, negative control or buffer blank, ten insects in each case, and evaluated after 1 hour and 10 hours.

TABLE 16
Efficacy of N-Acetyl Cysteine and Pyrrolidine
Carboxylic Acid supplemented Active Emulsions
against Cockroach Blata Orientalis

| | | | | |
|---|---|---|---|---|
| Sample/ | | | | |
| Exposure time | Immobile | Moribund | Alive | Mortality |

0.1% N-Acetyl Cysteine Supplemented Reactive Emulsion in 50 mM
citrate buffer

| | | | | | |
|---|---|---|---|---|---|
| 1 | hour | 2 | 2 | 6 | 40% |
| 10 | hours | 2 | 4 | 4 | 60% |

0.1% N-acetyl Cysteine in 50 mM citrate buffer

| | | | | | |
|---|---|---|---|---|---|
| 1 | hour | 0 | 0 | 10 | 0% |
| 10 | hours | 1 | 0 | 9 | 10% |

0.1% Pyrrolidone Carboxylic Acid supplemented reactive Emulsion in
50 mM citrate buffer

| | | | | | |
|---|---|---|---|---|---|
| 1 | hour | 0 | 6 | 4 | 60% |
| 10 | hours | 0 | 8 | 2 | 80% |

0.1% Pyrrolidone Carboxylic Acid in 50 mM citrate buffer

| | | | | | |
|---|---|---|---|---|---|
| 1 | hour | 0 | 0 | 10 | 0% |
| 10 | hours | 0 | 2 | 8 | 20% |

Control 5% Reactive Emulsion in 50 mM citrate buffer

| | | | | | |
|---|---|---|---|---|---|
| 1 | hour | 1 | 1 | 8 | 20% |
| 10 | hours | 0 | 3 | 7 | 30% |

50 mM Citrate Buffer Blank

| | | | | | |
|---|---|---|---|---|---|
| 1 | hour | 0 | 0 | 10 | 0% |
| 10 | hours | 0 | 1 | 9 | 10% |

A 5% W/W dispersion of reduced fatty acid Reactive Emulsion in 50 mM citrate buffer was shown to have 20% efficacy in 1 hour and 30% efficacy after 10 hours.

When supplemented with 0.1% NAC efficacy was increased to 40% after 1 hour and 60% after 10 hours.

0.1% NAC in Buffer without Reactive Emulsion had no effect in 1 hour and an apparent 10% efficacy after 10 hours.

Supplementation of Reactive Emulsion with 0.1% PCA increased efficacy to 60% and 80% after 1 hour and 10 hours respectively, while 0.1% PCA in buffer achieved just 20% efficacy after 10 hours.

### Example 14: Efficacy Against Mosquito

Colonies of mosquito were maintained in a glass enclosure. Because of the small size and erratic behaviour of individual insects it proved impossible to select exact numbers to conduct empirical testing.

The repellent effect of the Reactive Emulsion from Table 1 was demonstrated by placing cotton gauze patches saturated with 5% aqueous dispersion of emulsion at the bottom of the enclosure. Similar gauze patches saturated with water were placed at some 20 inches distance.

Observation showed a ‘halo’ effect over the reactive emulsion gauze where swarms of mosquito approached to approximately 6 inches and retreated. In contrast individual insects were landing on the water gauze and leaving with no apparent distress.

A 5% dispersion of the Reactive Emulsion from Table 1 was sprayed randomly into the glass enclosure and evaluated after 10 minutes. A significant number of dead and dying insects were observed on the floor of the enclosure but their relative proportion to living insects could not be counted reliably.

### Example 15: Safety on Human Skin Contact

A 5% dispersion of the Reactive Emulsion in Table 1 was subjected to European Standard ISO 10993-23 (2021) testing in a Certified independent laboratory. Following a battery of cell toxicity testing the formulation was applied to the forearms of 30 human volunteers (20 women, 10 men aged between 18 and 59 years)

As required under the Standard procedure there were three applications of the same formulation at 0, 24 and 48 hours under an occlusive patch with observations over that period and up to 72 hours after first application with no irritation or other deleterious effect detected.

### Example 16: Compositions of Free Fatty Acids

Fatty Acids with carbon chain length from 4 to 22 were investigated. If the Fatty Acid melting point is greater than physiological temperatures at the site of action, the oil phase will be a solid and will react very slowly to any pH change which will in turn limit the rate of collapse and delivery of phase inverting amphipath.

In general, as carbon chain length increases in saturated free fatty acids the melting point becomes progressively higher. The inclusion of one or more unsaturated carbon moieties within the chain length will significantly decrease the melting point in comparison to a saturated molecular entity of similar carbon number.

A combination of a low and high melting point fatty acid will depress the melting point of the higher entity and so compositions of free fatty acids with combined melting point approximating to the physiological temperature of the site of action may be generated.

Table 17 provides a range of free fatty acid with utility in this invention together with their individual melting points.

TABLE 17
Examples of fatty acids and combinations
with utility in this invention

| | | | Saturated/ |
|---|---|---|---|
| Common name | Carbon number* | Melting Point °C | Unsaturated |
| Butyric | 4 | −7.9 | Saturated |
| Caproic | 6 | −3.4 | Saturated |
| Caprylic | 8 | 16 | Saturated |
| Pelargonic | 9 | 12.5 | Saturated |
| Capric | 10 | 28 | Saturated |
| Undecylenic | 11 | 23 | Saturated |
| Lauric | 12 | 44 | Saturated |
| Myristic | 14 | 58 | Saturated |
| Palmitic | 16 | 63 | Saturated |
| Palmitoleic | 16:1 | −0.1 | Unsaturated |
| Stearic | 18 | 70 | Saturated |
| Oleic | 18:1 | 13 | Unsaturated |
| Linoleic | 18:2 | −5 | Unsaturated |
| Linolenic | 18:3 | −11 | Unsaturated |
| Arachidic | 20 | 76 | Saturated |
| Arachidonic | 20:4 | −50 | Unsaturated |
| Eicosapentanoic | 20:5 | −54 | Unsaturated |
| Behenic | 22 | 80 | Saturated |

It should be noted that the 16 carbon saturated palmitic acid has a melting point of 63 °C. A similar 16 carbon unsaturated palmitoleic acid with one unsaturated carbon bond has a melting point of −0.1 °C. Similarly the saturated 18 carbon Stearic acid has a melting point of 70 °C, but adding one, two or three unsaturated bonds (Oleic to linolenic) progressively decreases melting point to −11 °C. A combination of equal parts of stearic and linolenic acid has a combined melting point of 35 °C, which can be decreased further by increasing the relative concentration of linolenic. Equally low melting point triglyceride oils such as Miglyol 812 with a melting point of 6 °C can be used in combination with high melting point fatty acids to reduce the combined melting point to useable physiological temperatures.

Reduction of melting point using combinations of free fatty acids is disclosed in more detail in US2010/0317734 the entire contents of which is incorporated here by reference.

The insecticidal effect of combinations of high and low melting point fatty acids was illustrated using equal parts of lauric and palmitoleic acid to replace the 10% oil phase in Table 2. The combination had a melting point of 18 °C.

Insecticidal efficacy was demonstrated against the cockroach Blata Orientalis using the method described in Example 1, part 1 and a 10% dispersion of the Lauric/Palmitoleic reactive emulsion in water.

One hour after exposure 5 of the 10 test insects were dead and 5 were deemed to be moribund. After 10 hours 8 were dead and 2 remained moribund. All of the 10 control group treated with water were alive after 10 hours.

A similar combination of equal parts Myristic and Linolenic acid with a combined melting point of 20 °C was used to replace the 10% oil phase in Table 2 and an emulsion prepared as described in that Table. In a similar manner to the Lauric/Palmitoleic emulsion the insecticidal efficacy of the Myristic/Linolenic Reactive Emulsion was demonstrated using Blata Orientalis as described in Example 1 part 1.

After 1 hour 3 of the test insects were dead, 5 were moribund and 2 were alive. 10 hours after exposure 6 were dead, 2 were deemed to be moribund and 2 remained alive. All of the 10 water treated control group were alive after 10 hours.

From these results it would appear that a Reactive Emulsion based on lauric/Palmitoleic combination was more potent than one based on Myristic/linolenic, but further optimisation of the ratio could be expected to improve potency of both.

It will be appreciated that various features of the invention which are, for clarity, described in the contexts of separate embodiments may also be provided in combination in a single embodiment. Conversely, various features of the invention which are, for brevity, described in the context of a single embodiment may also be provided separately or in any suitable sub-combination. It will also be appreciated by persons skilled in the art that the present invention is not limited by what has been particularly shown and described hereinabove. Rather the scope of the invention is defined only by the claims which follow.

What is claimed is:

1. A composition comprising a reactive emulsion which comprises
a. an effective amount of one or more saturated or unsaturated free fatty acids having from 4 to 22 carbon atoms or an acceptable salt or ester thereof;
b. one or more membrane lipids or a hydrolysed derivative thereof, as an emulsifying agent for the free fatty acid(s), or the salt or ester thereof; and
c. a diluent or carrier;
wherein the fatty acid and membrane lipid form droplets, wherein the mean droplet size is less than 1 micron.

2. The composition according to claim 1, wherein the free fatty acid is selected from valeric, caproic, caprylic, pelargonic, capric, undecanoic, undecylenic, lauric, myristic, palmitic, stearic, oleic, linoleic and linolenic acids and mixtures thereof, and pharmaceutically acceptable salts and esters thereof.

3. The composition according to claim 2, wherein the free fatty acid is selected from one or more of caproic, caprylic, pelargonic, capric, undecylenic and lauric acids, especially caprylic acid.

4. The composition according to claim 3, wherein the free fatty acid is selected from caprylic and capric acids.

5. The composition according to claim 4, wherein the free fatty acid is caprylic acid.

6. The composition according to any preceding claim, wherein the membrane lipid is selected from one or more of phospholipids, lecithin, glycerophospholipids, sphingolipids, glycosphingolipids, glycoglycerolipids and cholesterols, and hydrolysed derivatives thereof.

7. The composition according to claim 6, wherein the membrane lipid is selected from one or more of phosphatidic acid, phosphatidylcholine, phosphatidylethanolamine, phosphatidylglycerol, phosphatidylinositol, phosphatidylserine, lecithin, ceramide, sphingomyelin, glycolipids, glycosphingolipids, cerebrosides, gangliosides, glycoglycerolipids, mono-galactosyl diglyceride, lanosterol and cholesterol.

8. The composition according to claim 7, wherein the membrane lipid is a phospholipid selected from one or more of phosphatidic acid, phosphatidylcholine, phosphatidylethanolamine, phosphatidylglycerol, phosphatidylinositol, phosphatidylserine and lecithin, especially lecithin.

9. The composition according to any preceding claim, wherein the membrane lipid or hydrolysed derivative thereof is de-lipidised.

10. The composition according to any preceding claim, wherein the membrane lipid or hydrolysed derivative thereof is de-lipidised lecithin.

11. The composition according to claim 10 wherein the de-lipidised lecithin contains less than 3% conjugated extraneous lipid material.

12. The composition according to any preceding claim, comprising a free fatty acid selected from one or more of caproic, caprylic, pelargonic, capric, undecylenic and lauric acids, in combination with de-lipidised lecithin.

13. The composition according to any preceding claim, wherein the weight ratio of fatty acids to membrane lipids is from about 0.25:1 to about 10:1; or from about 0.5:1 to about 10:1, or from about 0.5:1 to about 5.0:1, or from about 1.0:1 to about 2.5:1, or from about 1.25:1 to about 2.5:1, on a weight for weight basis.

14. The composition according to claim 1 wherein the composition comprises (a) one or more free fatty acids selected from the group consisting of caproic, caprylic, capric, lauric, palmitic, stearic, oleic, linoleic and linolenic acids and mixtures thereof; and
(b) one or more membrane lipids as emulsifying agent for the free fatty acid(s), wherein the membrane lipid is delipidized lecithin, wherein the weight ratio of component (a) to component (b) is from about 0.25:1 to about 10:1.

15. The composition according to any preceding claim, further comprising one or more pharmaceutically acceptable organic acids or a pharmaceutically acceptable salt or ester thereof; and/or one or more pharmaceutically acceptable organic acid salts.

16. The composition according to claim 5 wherein the organic acid is selected from acetic, pyruvic, propionic, glycolic, oxalic, lactic, glyceric, tartronic, malic, maleic, ascorbic, fumaric, tartaric, malonic, glutaric, propenoic, cis or trans butanoic and citric acids and mixtures thereof, and pharmaceutically acceptable salts and esters thereof.

17. The composition according to claim 16, wherein the organic acid is citric or lactic acid or the sodium or potassium salt thereof.

18. The composition according to claim 15, wherein the organic acid salt is sodium citrate.

19. The composition according to claim 15, wherein the organic acid salt is selected from sodium, potassium and calcium.

20. The composition according to any preceding claim, in the form of an oil-in-water reactive emulsion.

21. The composition according to claim 1, wherein the composition is formulated as a spray, aerosol, liquid, gel, powder, paste, ointment, cream, surface coating, soap, dry residue, lotion, or wipe.

22. A insecticidal and/or arachnidicidal composition comprising:
a. an effective amount of one or more saturated or unsaturated free fatty acids having from 4 to 22 carbon atoms or a pharmaceutically acceptable salt or ester thereof; and one or more membrane lipids or a hydrolysed derivative thereof, as emulsifying agent for the free fatty acid(s) or the salt or ester thereof; and
b. a diluent or carrier.

23. A method for the control of insects and/or arachnids, the method comprising contacting said insects and/or arachnids, or a locus at which control is desired, with an effective amount of a composition of any of the above claims.

24. The method of claim 23, wherein the insect and/or arachnid is a flea, midge, mosquito, horse-fly, tsetse fly, sandfly, blackfly, bedbug, assassin bug, gnat, louse, sawfly, ant, caterpillar, aphid, locust, weevil, beetle, whitefly, thrip, scale, earwig, capsid, mealybug, spider, tick, mite, scorpion, pseudoscorpion, harvestmen, camel spider, whip spider, or vinegarroon.

25. The method of any of claims 23-24, wherein the locus is a subject or a plant.

26. The method of claim 25, wherein the locus is a plant.

27. The method of claim 26, wherein the plant is a feed crop (fruit, vegetable or grain), forage crop (grasses), fiber crop (cotton, hemp or flax), oil crop (canola, olive, soybean or corn), ornamental crop (flowers, hedges, trees), or industrial crop (rubber or tobacco).

28. The method of any one of claims 26-27, wherein the insect and/or arachnid is a caterpillar, aphid, locust, weevil, beetle, whitefly, thrip, scale, earwig, capsid, or mealybug.

29. The method of claim 25, wherein the locus is a subject.

30. The method of claim 29, wherein the subject is a mammal.

31. The method of claim 30, wherein the subject is human or non-human mammal.

32. The method of claim 29, wherein the subject is a household pet, farm animal or zoo animal.

33. The method of claim 32, wherein the subject is a human, dog, cat, mouse, rat, gerbil, ferret, hamster, bird, horse, pony, donkey, mule, lama, alpaca, emu, sheep, goat, pig, cow, bull, steer, heifer, deer, tiger, cheetah, wolf, monkey, lion, bear, fox, gorilla, or kangaroo.

34. The method of any one of claims 29-33, wherein the insect and/or arachnid is a flea, midge, mosquito, horse-fly, tsetse fly, sandfly, blackfly, bedbug, assassin bug, gnat, louse, sawfly, ant, spider, tick, mite, scorpion, pseudoscorpion, harvestmen, camel spider, whip spider, or vinegarroon.

35. The method of claim 34, wherein the insect is a flea, midge, mosquito, horse-fly, bedbug, gnat, louse, ant, spider, tick, or mite.

36. The method of claim 35, wherein the insect is a flea, midge, mosquito, horse-fly, gnat, louse, tick, or mite.

37. The method of any one of claims 29-33, wherein the insect and/or arachnid is a hematophagous organism.

38. The method of any one of claims 23-37 wherein the compositions are contacted with said locus prior to an infestation by said insects and/or arachnids.

39. The method of any one of claims 23-37 wherein the compositions are contacted directly with said insect and/or arachnid.

40. The method of any one of claims 23-37 wherein the compositions are formulated as a spray, aerosol, liquid, gel, powder, paste, ointment, cream, surface coating, soap, dry residue, lotion or wipe.

41. The method of any one of claims 23-40 wherein the compositions are contacted with said insects and/or arachnids or said locus daily, weekly, bi-weekly, monthly or annually.

42. The method of any one of claims 29-41, wherein the insect is a mosquito.

43. The method of any one of claims 29-41, wherein the insect is a tick.

44. A method of treating and/or preventing a physiological and/or an immunological response in a subject in need thereof, wherein said physiological and/or an immunological response is caused by a bite or a sting from an insect and/or arachnid, the method comprising contacting said insects and/or arachnids, said subject, or a locus at which treatment and/or prevention is desired, with a therapeutically effective amount of a composition of any one of claims 1-22; and a pharmaceutically acceptable diluent or carrier.

45. The method of claim 44, wherein the insect and/or arachnid is a flea, midge, mosquito, horse-fly, tsetse fly, sandfly, blackfly, bedbug, assassin bug, gnat, louse, sawfly, ant, spider, tick, mite, scorpion, pseudoscorpion, harvestmen, camel spider, whip spider, or vinegarroon.

46. The method of claim 45, wherein the insect is a flea, midge, mosquito, horse-fly, bedbug, gnat, louse, ant, spider, tick, or mite.

47. The method of claim 46, wherein the insect is a flea, midge, mosquito, horse-fly, gnat, louse, tick, or mite.

48. The method of claim 44, wherein the insect and/or arachnid is a hematophagous organism.

49. The method of any one of claims 44-48, wherein the subject is a mammal.

50. The method of claim 49, wherein the subject is human or non-human mammal.

51. The method of any one of claims 44-48, wherein the subject is a household pet, farm animal or zoo animal.

52. The method of any one of claims 44-48, wherein the subject is a human, dog, cat, mouse, rat, gerbil, ferret, hamster, bird, horse, pony, donkey, mule, lama, alpaca, emu, sheep, goat, pig, cow, bull, steer, heifer, deer, tiger, cheetah, wolf, monkey, lion, bear, fox, gorilla, or kangaroo.

53. The method of any one of claims 44-52, wherein the compositions are contacted with said insects and/or arachnids, said subject, or said locus at which treatment and/or prevention is desired, prior to an infestation by said insects and/or arachnids.

54. The method of any one of claims 44-52, wherein the compositions are contacted directly with said insect and/or arachnid.

55. The method of any one of claims 44-52, wherein the compositions are contacted directly with said subject.

56. The method of any one of claims 44-54 wherein the compositions are formulated as a spray, aerosol, liquid, gel, powder, paste, ointment, cream, surface coating, soap, dry residue, lotion or wipe.

57. The method of any one of claims 44-56 wherein the compositions are contacted with said insects and/or arachnids or said locus daily, weekly, bi-weekly, monthly or annually.

58. The method of any one of claims 44-57 wherein said physiological and/or an immunological response is inflammation; itchiness; erythema; hives; redness; pain; vasoconstriction; platelet aggregation; degranulation of mast cells; release of dendritic cells; activation of fibroblasts, fibrinogen, bradykinins, histamines, or chemoattractant (chemokines or leukotrienes); transport of neutrophils or monocytes; or generating antigen specific antibodies or T lymphocytes.

59. The composition of claim 1 or 22, further comprising an amino acid derivative.

60. The composition of claim 59, wherein the amino acid derivative is N-Acetyl Cysteine or Pyrrolidone Carboxylic Acid/Pyroglutamic Acid or combinations thereof.

61. The composition of claim 59 or 60, wherein the amino acid derivative comprises between about 0.1% and about 10%, between about 0.5% and about 5%, or between about 1% and about 2% w/w of the water phase.
